(19) United States
(12) Patent Application Publication
(10) Pub. No.: US 2009/0221568 A1
(43) Pub. Date: Sep. 3, 2009

(54) Synthesis of Inhibitors of FtsZ

(76) Inventors: Jared Shaw, Davis, CA (US); Sameer Urgaonkar, Cambridge, MA (US); Debabrata RayChaudhuri, Somerville, MA (US); Henry La Pierre, Berkeley, CA (US)

Correspondence Address: CHOATE, HALL & STEWART/HARVARD UNIVERSITY, TWO INTERNATIONAL PLACE, BOSTON, MA, 02110, US

(21) Appl. No.: 12/092,391

(22) PCT Filed: Nov. 3, 2006
(86) PCT No.: PCT/US2006/043042

§ 371 Date: Dec. 18, 2008

Related U.S. Application Data
(60) Provisional application No. 60/733,543, filed on Nov. 4, 2005.

(51) Int. Cl. A61K 31/5377; C07D 311/30; A61K 31/352; C07D 413/14; A61P 31/04; C12Q 1/18

(52) U.S. Cl. 514/232.5; 549/403; 514/456; 544/79; 435/32

(57) ABSTRACT

FtsZ, the bacterial analog of tubulin, is a promising new target for developing new antibiotics. It has been shown that polyphenols inhibit the GTPase activity of FtsZ, thereby inhibiting Z-ring formation during mitosis. The present invention provides novel polyphenols compounds, which can be accessed by the synthesis of dichamametin and 2′″-hydroxy-5″-benzylisouvarinol-B as described herein. These novel compounds are useful in treating infections, particularly infections caused by gram-positive organisms. Methods of preparing the inventive compounds are also provided. The compounds are prepared by the benzylation of pinocembrin or chrysin core structure. Pharmaceutical compositions and method of using the compounds to treat disease are also provided. These compounds may be screened for antimicrobial activity as well as other biological activities such as anti-neoplastic, anti-inflammatory, immunosuppressive, and cytotoxic activity.

### RELATED APPLICATIONS

The present application claims priority under 35 U.S.C. § 119(e) to U.S. provisional patent application, U.S. Ser. No. 60/733,543, filed Nov. 4, 2005, which is incorporated herein by reference.

### GOVERNMENT SUPPORT

The work described herein was supported, in part, by grants from the National Institutes of Health (National Institute of Allergy and Infectious Disease, R03 AI062905-01; and P50 GM069721). The United States government may have certain rights in the invention.

### BACKGROUND OF THE INVENTION

The emergence of bacterial strains that are resistant to current drugs has prompted a renewed effort to discover new methods for fighting infectious disease. Walsh, Nat. Rev. Microbiol. 2003, 1, 65-70; Walsh et al. Chem. Rev. 2005, 105, 391-393; each of which is incorporated herein by reference. One promising new target is FtsZ, the bacterial analog of tubulin, which mediates bacterial cell division. During bacterial cytokinesis, FtsZ monomers polymerize at mid-cell to form the Z-ring, which eventually constricts, leading to septation and formation of daughter cells (FIG. 1). FtsZ consumes GTP during Z-ring assembly, much like its eukaryotic analog tubulin during mitosis. As such, FtsZ is susceptible to inactivation by compounds that interfere with the GTPase activity of this protein. Romberg et al. Ann. Rev. Microbiol. 2003, 57, 125-154; Margolin, Curr. Biol. 2003, 13, R16-R18; Addinall et al. J. Mol. Biol. 2002, 318, 219-236; Weiss, Mol. Microbiol. 2004, 54, 588-597; Errington et al. Microbiol. Mol. Biol. Rev. 2003, 67, 52-65; each of which is incorporated herein by reference.

Recent studies have revealed several compounds that inhibit the GTPase activity of FtsZ and kill bacteria (Wang et al. J. Biol. Chem. 2003, 278, 44424-44428; Reynolds et al. Bioorg. Med. Chem. Lett. 2004, 14, 3161-3164; White et al. J. Antimicrob, Chemother. 2002, 50, 111-114; each of which is incorporated herein by reference), and in one case several compounds were demonstrated to disrupt Z-ring formation. Margalit et al. Proc. Nat'l. Acad. Sci. U.S.A. 2004, 101, 11821-11826; the antibacterial activity of this compound was first noted by Beaver: Beaver et al. J. Am. Chem. Soc. 1953, 75, 5579-5581; subsequent to Beaver, Hakimelahi made similar observations with 1 and related analogs: Hakimelahi et al. Helv. Chim. Acta 1981, 64, 599-609; Moshfegh et al. Helv. Chim. Acta 1982, 65, 1221-1228; each of which is incorporated herein by reference. Zantrin Z1 (1, FIG. 2), which was discovered in a high throughput in vitro screen for inhibition of GTPase activity, possesses a polyphenolic structure reminiscent of several natural products that exhibit potent antimicrobial activity. Dichamanetin (2) and 2′″-hydroxy-5″-benzylisouvarinol-B (3), isolated independently by Hufford and Anam from U. chamae and X. afticana respectively, exhibit comparable MIC values to zantrin Z1 when evaluated against a variety of bacterial strains. Hufford et al. Lloydia 1978, 41, 156-160; Anam, Ind. J. Chem., Sect. B 1994, 33B, 1009-1011; each of which is incorporated herein by reference. It is notable that these compounds show a high level of activity against gram positive bacteria (e.g. S. aureus and B. subtilis), and furthermore the MIC values are comparable to clinically relevant antibiotics. (The National Committee for Clinical Laboratory standards (NCCLS) defines the effective MICs of methicillin, erythromycin, and vancomycin as 0.8, 0.4, and 5.8 μM respectively. Higher MICs indicate that a strain is becoming resistant to the drug. Leegaard et al. Clin. Microbiol. Infect. 2000, 6, 290-293; incorporated herein by reference.)

There remains a need for a practical and efficient synthetic route to dichamanetin, 2′″-hydroxy-5″-benzylisouvarinol-B, and analogs thereof. Novel analogs of dichamanetin and 2′″-hydroxy-5″-benzylisouvarinol-B may be useful in the treatment of infection.

### SUMMARY OF THE INVENTION

The present invention stems from the recognition that zantrin 1, dichamanetin, and 2′″-hydroxy-5″-benzylisouvarinol-B are structurally similar suggesting that they might all derive their antimicrobial activity by inhibiting the GTPase activity of FtsZ. While naturally-occurring flavanones have attracted the attention of synthetic chemists and biologists alike, benzylated flavanones are quite rare, and as such no efficient syntheses of compounds related to dichamanetin and 2′″-hydroxy-5″-benzylisouvarinol-B have been reported. (The synthesis of gericudranin, a relatedpara-hydroxybenzylated 3-hydroxy flavanone has been reported: Choi et al. Heterocycles 1996, 43, 1223-1228) Therefore, an efficient synthesis of dichamanetin and 2′″-hydroxy-5″-benzylisouvarinol-B was developed. The synthesis begins with pinocembrin or an analog thereof. The pinocembrin core is then functionalized to yield novel compounds. The synthesis is amenable to preparing novel analogs of dichamanetin and 2′″-hydroxy-5″-benzylisouvarinol-B. These analogs can be tested for novel biological activities including the ability to inhibit the GTPase activity of FtsZ, inhibit bacterial growth, and cytotoxic activity.

Inventive compounds which are accessible via the synthetic route are typically of the formula:

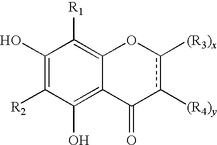

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; ═O; —C(═O)R_{A}; —CO₂R_{A}; —CN; —SCN; —SRA; —SORA; —SO₂R_{A}; —NO₂; —N(R_{A})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or tnsubstituted, branched or unbranched acyl; substituted or unsubstituted, branched or unbranched aryl; substituted or unsubstituted, branched or unbranched heteroaryl; —OR_{B}; ═O; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, allylamino, diallylamino, heteroaryloxy; or heteroarylthio moiety; and

each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; ═O; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of P is independently hydrogen, substituted or unsubstituted aliphatic, substituted or unsubstituted heteroaliphatic, a protecting group, substituted or unsubstituted acyl, substituted or unsubstituted aryl, or substituted or unsubstituted heteroaryl;

x is 1 or 2, wherein if the dashed line is a bond, x is 1;

y is 1 or 2, wherein if the dashed line is a bond, y is 1;

the dashed line represents the presence or absence of a bond; and

derivatives, salts, pro-drugs, and isomers thereof.

A subclass of the inventive compounds is of the formula:

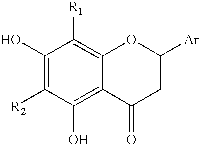

wherein Ar is a substituted or unsubstituted aryl or heteroaryl group.

Another subclass of the inventive compounds is of the formula:

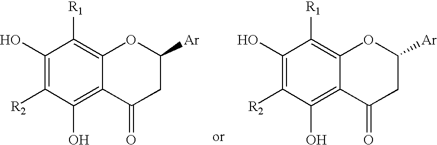

wherein Ar is a substituted or unsubstituted aryl or heteroaryl group.

A subclass of the inventive compounds is of the formula:

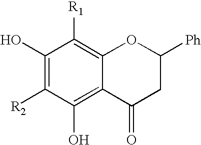

wherein Ph is a substituted or unsubstituted phenyl group.

Another subclass of the inventive compounds is of the formula:

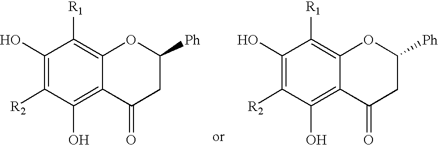

wherein Ph is a substituted or unsubstituted phenyl group.

A further subclass of the inventive compounds is of the formula:

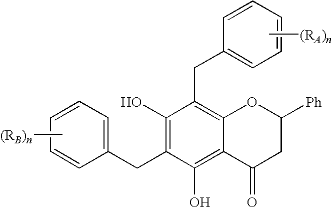

wherein

each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; C(═O)R_{A}′; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

each occurrence of n is an integer between 1 and 5, inclusive.

A further class of the inventive compounds includes compounds of the formula:

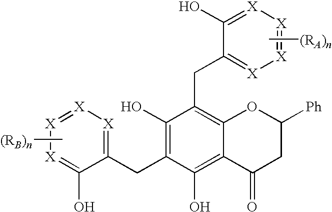

wherein

X is C or N and there are no more than three N atoms in any ring;

each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

each occurrence of n is an integer between 1 and 5, inclusive.

Particular compounds of the invention include:

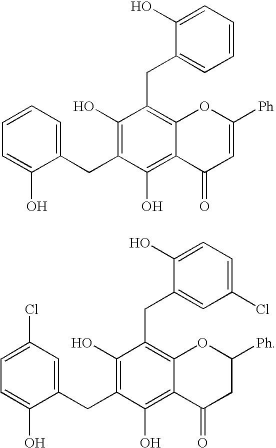

The present invention also provides methods of preparing the compounds of the invention. An exemplary synthesis of the inventive compounds is shown below:

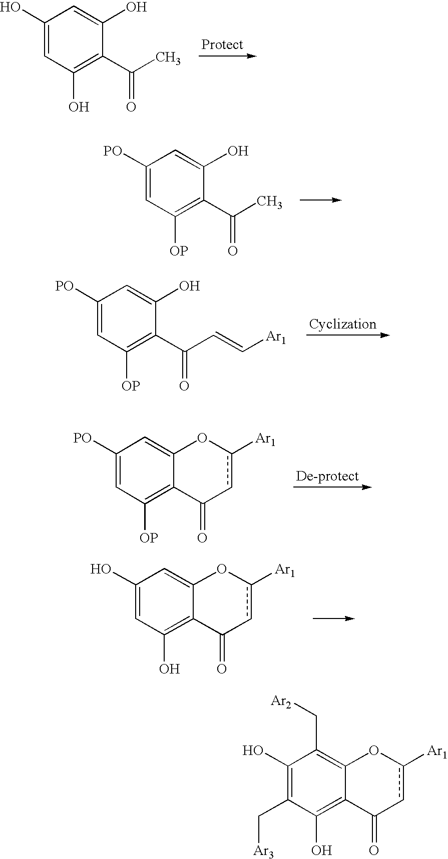

The first four steps of the synthesis provide the chrysin or pinocembrin core of the inventive compound. In certain embodiments, the inventive compounds are prepared from chrysin or pinocembrin. In other embodiments, the inventive compounds are prepared from a derivative of chrysin or pinocembrin. An exemplary synthesis of pinocembrin is shown in FIG. 4. As will be appreciated by one of skill in the art, derivatives of pinocembrin or chrysin may be prepared using the a similar synthetic route. The pinocembrin and chrysin core is then functionalized to yield the inventive compounds. In certain embodiments, the core is functionalized using carbon-carbon coupling reactions. The coupling reactions are used to functionalize the aromatic ring of the core with cyclic, heterocyclic, aryl, and heteroaryl substituents. The substituents may be substituted or unsubstituted, branched or unbranched. In certain embodiments, the inventive compounds is prepared using a benzylic coupling reaction via ortho quinone methide intermediates (see FIGS. 6-8). The benzylic coupling reaction is performed in the presence of a Lewis or protic acid. In certain embodiments, Ar₂ and Ar₃ are the same. In other embodiments, Ar₂ and Ar₃ are different.

The present invention also includes all intermediates useful in the synthesis of compounds of the present invention. These intermediates include pinocembrin, derivatives of pinocembrin, chrysin, derivative of chrysin, and reagents for functionalizing the chrysin and pinocembrin core (e.g., ortho quninone methide intermediates). The intermediates include various substituted forms, isomers, stereoisomers, salts, and derivatives thereof.

In one aspect of the invention, a compound that is useful as an intermediate in the synthesis of the inventive compounds is of the formula:

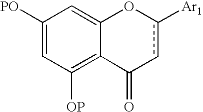

wherein

each occurrence of P is independently hydrogen, substituted or unsubstituted aliphatic, substituted or unsubstituted heteroaliphatic, a protecting group, substituted or unsubstituted acyl, substituted or unsubstituted aryl, or substituted or unsubstituted heteroaryl; and

Ar₁ is a substituted or unsubstituted aryl or heteroaryl group.

In another aspect, the present invention provides methods of treatment and pharmaceutical compositions comprising the inventive compounds. The pharmaceutical compositions may optionally include a pharmaceutically acceptable excipient. The methods and pharmaceutical compositions may be used to treat any infections. The inventive compounds are particularly useful in treating infections caused by gram positive bacteria. In certain embodiments, the infections are caused by antibiotic-resistant organisms. In certain instances, the compounds of the invention exhibit anti-neoplastic or anti-proliferative activity, in which case the compounds may be useful in the treatment of diseases such as cancer, autoimmune diseases, inflammatory diseases, and diabetic retinopathy. The methods and compositions may be used to treat disease in humans and other animals including domesticated animals. Any mode of administration including oral and parenteral administration of the pharmaceutical composition may be used. The inventive compounds may also be prepared in extended release formulations.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxylprotecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkylp-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynli carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkoxy”, or “thioalkyl” as used herein refers to an alkyl group, as previously defined, attached to the parent molecule through an oxygen atom or through a sulfur atom. In certain embodiments, the alkyl, alkenyl, and alkynyl groups contain 1-20 alipahtic carbon atoms. In certain other embodiments, the alkyl, alkenyl, and alkynyl groups contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “allylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoallyl groups include, but are not limted to, aziridinyl, pyrrolidinyl, piperidinyl, morpholinyl, pyrrolyl, imidazolyl, 1,3,4-trianolyl, and tetrazolyl.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ, independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Carbocycle”: The term “carbocycle”, as used herein, refers to an aromatic or non-aromatic ring in which each atom of the ring is a carbon atom.

“Independently selected”: The term “independently selected” is used herein to indicate that the R groups can be identical or different.

“Labeled”: As used herein, the term “labeled” is intended to mean that a compound has at least one element, isotope, or chemical compound attached to enable the detection of the compound. In general, labels typically fall into three classes: a) isotopic labels, which may be radioactive or heavy isotopes, including, but not limited to, ²H, ³H, ³²P, ³⁵S, ⁶⁷Ga, ⁹⁹ mTc (Tc-99m), ¹¹¹In, ¹²³I, ¹²⁵I, ¹⁶⁹Yb and ¹⁸⁶Re; b) immune labels, which may be antibodies or antigens, which may be bound to enzymes (such as horseradish peroxidase) that produce detectable agents; and c) colored, luminescent, phosphorescent, or fluorescent dyes. It will be appreciated that the labels may be incorporated into the compound at any position that does not interfere with the biological activity or characteristic of the compound that is being detected. In certain embodiments of the invention, photoaffinity labeling is utilized for the direct elucidation of intermolecular interactions in biological systems. A variety of known photophores can be employed, most relying on photoconversion of diazo compounds, azides, or diazirines to nitrenes or carbenes (See, Bayley, H., Photogenerated Reagents in Biochemistry and Molecular Biology (1983), Elsevier, Amsterdam.), the entire contents of which are hereby incorporated by reference. In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

“Tautomers”: As used herein, the term “tautomers” are particular isomers of a compound in which a hydrogen and double bond have changed position with respect to the other atoms of the molecule. For a pair of tautomers to exist there must be a mechanism for interconversion. Examples of tautomers include keto-enol forms, imine-enamine forms, amide-imino alcohol forms, amidine-aminidine forms, nitroso-oxime forms, thio ketone-enethiol forms, N-nitroso-hydroxyazo forms, nitro-aci-nitro forms, and pyridione-hydroxypyridine forms.

Definitions of non-chemical terms used throughout the specification include:

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.

“Effective amount”: In general, the “effective amount” of an active agent or the microparticles refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient. For example, the effective amount of an inventive compound is the amount that results in a sufficient concentration at the site of the infection to kill the microorganism causing the infection (bacteriocidal) or to inhibit the reproduction of such microorganisms (bacteriostatic). In another example, the effective amount of an inventive compound is the amount sufficient to reverse clinicals signs and symptoms of the infection, including fever, redness, warnth, pain, chills, cultures, and pus production.

### BRIEF DESCRIPTION OF THE DRAWING

FIG. 1 illustrates bacterial cell division and the role of FtsZ.

FIG. 2 depicts the structures of synthetic zantrin Z1 (1) and naturally occurring phenolic compounds, dichamanetin (2) and 2′″-hydroxy-5″-benzylisouvarinol-B (3).

FIG. 3 shows two retrosynthetic analyses of dichamanetin (2) and 2′″-hydroxy-5″-benzylisouvarinol-B (3).

FIG. 4 is a scheme showing the synthesis of pinocembrin (4).

FIG. 5 shows the aminomethylation of pinocembrin (4) and the attempted displacement with phenol to produce dichamanetin (2).

FIG. 6 is a table illustrating the optimization of the benzylation conditions using various protic and Lewis acids. The benzylation reaction is shown at the top of the table.

FIG. 7 shows the benzylation of pinocembrin (4) in the presence of the Lewis acid ZnCl₂ to produce dichamanetin (2).

FIG. 8 is a scheme showing the synthesis of 2′″-hydroxy-5″-benzylisouvarinol-B (3).

FIG. 9 is a ¹H NMR spectrum of 7, a protected form of pinocembrin (4).

FIG. 10 is a ¹³C NMR spectrum of 7, a protected form of pinocembrin (4).

FIG. 11 is a ¹H NMR spectrum of the morpholine (8).

FIG. 12 is a ¹³C NMR spectrum of the morpholine (8).

FIG. 13 is a ¹H NMR spectrum of the dimethylamine (9).

FIG. 14 is a ¹H NMR spectrum of the polyphenol (11).

FIG. 15 is a ¹³C NMR spectrum of the polyphenol (11).

FIG. 16 is a ¹H NMR spectrum of the intermediate (13).

FIG. 17 is a ¹³C NMR spectrum of the intermediate (13).

FIG. 18 is a ¹H NMR spectrum of the intermediate (14).

FIG. 19 is a ¹³C NMR spectrum of the intermediate (14).

FIG. 20 is a ¹H NMR spectrum of dichamanetin (2).

FIG. 21 is a ¹³C NMR spectrum of the dichamanetin (2).

FIG. 22 is a ¹H NMR spectrum of a protected form of 2′″-hydroxy-5″-benzylisouvarinol-B (3).

FIG. 23 is a ¹³C NMR spectrum of a protected form of 2′″-hydroxy-5″-benzylisouvarinol-B (3).

FIG. 24 is a ¹H NMR spectrum of 2′″-hydroxy-5″-benzylisouvarinol-B (3).

FIG. 25 is a ¹³C NMR spectrum of 2′″-hydroxy-5″-benzylisouvarinol-B (3).

FIG. 26 is a ¹H NMR spectrum of 2′″-hydroxy-5″-benzylisouvarinol-B (3) with two drops of D₂O added and the spectrum acquired 1 day later.

FIG. 27 is a scheme of the synthesis of a dimethyl achiral derivative of dichamanetin. This derivative has an IC₅₀ of 21 μM.

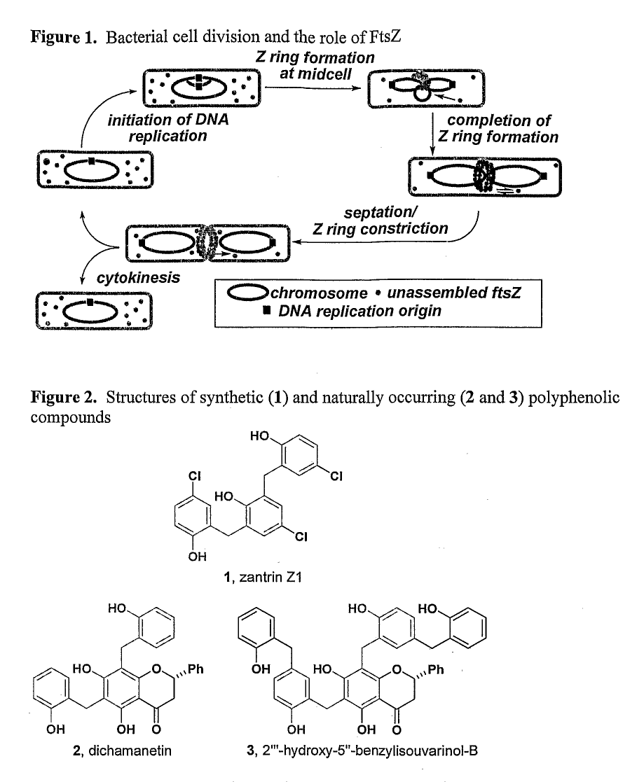

### DETAILED DESCRIPTION OF CERTAIN PREFERRED EMBODIMENTS OF THE INVENTION

The present invention stems from the development of a synthetic route that allows the preparation of polyphenol compounds. The synthesis has been used to prepare the naturally occurring compounds dichamanetin (2) and 2′″-hydroxy-5″-benzylisouvarinol-B (3). These compounds have been shown to inhibit the GTPase activity of FtsZ, the bacterial analog of tubulin which is important in bacterial cell division. By inhibiting Z-ring formation, these compounds have an anti-microbial effect. The synthetic route provides novel related compound. These new compounds may also exhibit anti-microbial activity. Other compounds accessible via the new synthetic route may exhibit other biological activities such as anti-neoplastic or anti-proliferative activity.

### Compounds

Compounds of the present invention include polyphenols. For example, derivatives of zantrin Z1 (1), dichamanetin (2), and 2′″-hydroxy-5″-benzylisouvarinol-B (3) are provided by the present invention. Particularly useful compounds of the present invention include those with biological activity. In certain embodiments, the compounds of the invention exhibit antimicrobial activity. In particular, certain compounds of the invention inhibit the GTPase activity of FtsZ. In certain embodiments, the compounds disrupt or inhibit Z-ring formation in bacteria. In certain embodiments, the compound have a mean inhibitory concentration, with respect to a particular bacteria, of less than 50 μg/mL, preferably less than 25 μg/mL, more preferably less than 5 μg/mL, and most preferably less than 1 μg/mL. For example, infection caused by the following organisms may be treated with antimicrobial compounds of the invention: Gram-positives—Staphylocococcus aureus, Streptococcus Group A, Streptococcus viridans, Streptococcus pneumoniae; Gram-negatives—Neisseria meningitidis, Neisseria gonorrhoeae, Haemophilus influenzae, Escherichia coli, Bacteroides fragilis, other Bacteroides; and Others—Mycoplasma pneumoniae, Treponema pallidum, Rickettsia, and Chlamydia. In other embodiments, the compounds of the invention exhibit antiproliferative, antineoplastic, anti-inflammatory, or immunosuppressive activity.

In certain embodiments, the compounds of the present invention are represented by the formula:

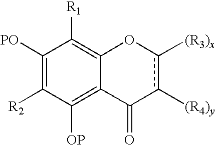

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; ═O; —C(═O)R_{A}; —CO₂R_{A}; —CN; —SCN; —SRA; —SORA; —SO₂R_{A}; —NO₂; —N(R_{A})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; ═O; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SRC; —SORC; —SO₂R_{C}; —NO₂; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; ═O; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of P is independently hydrogen, substituted or unsubstituted aliphatic, substituted or unsubstituted heteroaliphatic, a protecting group, substituted or unsubstituted acyl, substituted or unsubstituted aryl, or substituted or unsubstituted heteroaryl;

x is 1 or 2, wherein if the dashed line is a bond, x is 1;

y is 1 or 2, wherein if the dashed line is a bond, y is 1;

the dashed line represents the presence or absence of a bond; and

derivatives, salts, pro-drugs, and isomers thereof.

In certain embodiments, when the dashed line represents no bond, R₃ is phenyl, and R₄ is hydrogen, then R₁ and R₂ are not both selected from the group consisting of:

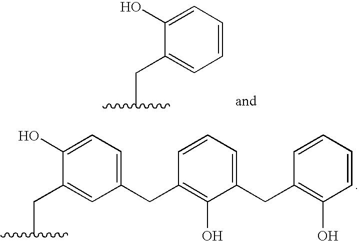

In other embodiments, when the dashed line represents no bond, R₃ is phenyl, and R₄ is hydrogen, then R₁ and R₂ are not both selected from the group consisting of:

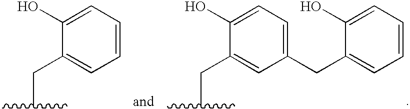

In still other embodiments, when the dashed line represents no bond, R₃ is phenyl, and R₄ is hydrogen, then R₁ and R₂ are not both selected from the group consisting of:

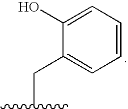

In certain embodiments, when the dashed line represents no bond, R₃ is phenyl, R₄ is hydrogen, and the carbon attached to R₃ is in the S-configuration, then R₁ and R₂ are not both selected from the group consisting of:

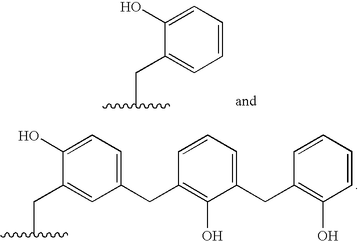

In other embodiments, when the dashed line represents no bond, R₃ is phenyl, R₄ is hydrogen, and the carbon attached to R₃ is in the S-configuration, then R₁ and R₂ are not both selected from the group consisting of:

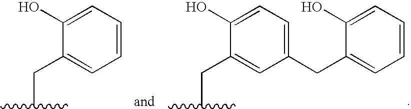

In yet other embodiments, when the dashed line represents no bond, R₃ is phenyl, R₄ is hydrogen, and the carbon attached to R₃ is in the S-configuration, then R₁ and R₂ are not both selected from the group consisting of:

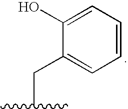

In certain embodiments, each occurrence of P is hydrogen or aliphatic. In certain embodiments, each occurrence of P is hydrogen or alkyl. In certain embodiments, each occurrence of P is hydrogen or C₁-C₆ alkyl. In other embodiments, each occurrence of P is hydrogen or acyl. In other embodiments, each occurrence of P is hydrogen or an oxygen protecting group. In other embodiments, each occurrence of P is hydrogen or a silicon-containing protecting group. In yet other embodiments, each occurrence of P is selected from hydrogen, methyl, methoxymethyl, benzyl, TBDMS, TMS, TES, acetyl, MS, and Ts. In certain embodiments, both occurrences of P are hydrogen. In other embodiments, both occurrences of P are methoxymethyl. In yet other embodiments, both occurrences of P are an oxygen-protecting group.

In certain embodiments, R₁ is a substituted or unsubstituted, branched or unbranched arylaliphatic moiety. In certain embodiments, R₁ is a substituted or unsubstituted, branched or unbranched arylalkyl moiety. In certain embodiments, R₁ is a substituted or unsubstituted, branched or tnbranched arylalkenyl moiety. In certain embodiments, R₁ is a substituted or unsubstituted, branched or unbranched arylalkynyl moiety. In other embodiments, R₁ is a substituted or unsubstituted, branched or unbranched heteroarylaliphatic moiety. In certain embodiments, R₁ is a substituted or unsubstituted, branched or unbranched heteroarylalkyl moiety. In certain embodiments, R₁ is a substituted or unsubstituted, branched or unbranched heteroarylalkenyl moiety. In certain embodiments, R₁ is a substituted or unsubstituted, branched or unbranched heteroarylalkynyl moiety. In certain embodiments, R₁ comprises a monocyclic ring system. In certain specific embodiments, R₁ comprises a 5-7-membered monocyclic ring system. In other embodiments, R₁ comprises a bicyclic ring system. In certain embodiments, R₁ comprises a bicyclic ring system with 5-membered or 6-membered rings.

In certain embodiments, R₁ is a substituted or unsubstituted benzyl moiety. In other embodiments, R₁ is a substituted benzyl moiety. In yet other embodiments, R₁ is a substituted benzyl moiety, substituted with at least one hydroxyl group.

In certain embodiments, R₁ is

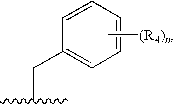

wherein each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and n is an integer between 1 and 5, inclusive. In certain embodiments, R₁ is

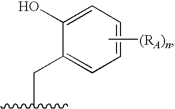

wherein each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and n is an integer between 1 and 5, inclusive. In certain embodiments, R₁ is

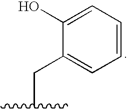

In other embodiments, R₁ is

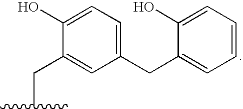

In yet other embodiments, R₁ is

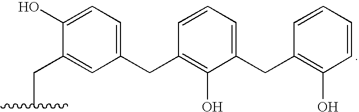

In certain embodiments, R₁ is a substituted or unsubstituted, branched or unbranched cyclicaliphatic moiety. In certain embodiments, R₁ is a substituted or unsubstituted, branched or unbranched cyclicalkyl moiety. In certain embodiments, R₁ is a substituted or unsubstituted, branched or unbranched cyclicalkenyl moiety. In certain embodiments, R₁ is a substituted or unsubstituted, branched or unbranched cyclicalkynyl moiety. In other embodiments, R₁ is a substituted or unsubstituted, branched or unbranched heterocyclicaliphatic moiety. In certain embodiments, R₁ is a substituted or unsubstituted, branched or unbranched heterocyclicalkyl moiety. In certain embodiments, R₁ is a substituted or unsubstituted, branched or unbranched heterocyclicalkenyl moiety. In certain embodiments, R₁ is a substituted or unsubstituted, branched or unbranched heterocylicalkynyl moiety. In certain embodiments, R₁ comprises a monocyclic ring system. In certain specific embodiments, R₁ comprises a 5-7-membered monocyclic ring system. In other embodiments, R₁ comprises a bicyclic ring system. In certain embodiments, R₁ comprises a bicyclic ring system with 5-membered or 6-membered rings.

In certain embodiments, R₂ is a substituted or unsubstituted, branched or unbranched arylaliphatic moiety. In certain embodiments, R₂ is a substituted or unsubstituted, branched or unbranched arylalkyl moiety. In certain embodiments, R₂ is a substituted or unsubstituted, branched or unbranched arylalkenyl moiety. In certain embodiments, R₂ is a substituted or unsubstituted, branched or unbranched arylallynyl moiety. In other embodiments, R₂ is a substituted or unsubstituted, branched or unbranched heteroarylaliphatic moeity. In certain embodiments, R₂ is a substituted or unsubstituted, branched or unbranched heteroarylalkyl moiety. In certain embodiments, R₂ is a substituted or unsubstituted, branched or unbranched heteroarylalkenyl moiety. In certain embodiments, R₂ is a substituted or unsubstituted, branched or unbranched heteroarylalkynyl moiety. In certain embodiments, R₂ comprises a monocyclic ring system. In certain specific embodiments, R₂ comprises a 5-7-membered monocyclic ring system. In other embodiments, R₂ comprises a bicyclic ring system. In certain embodiments, R₂ comprises a bicyclic ring system with 5-membered or 6-membered rings. In certain embodiments, R₂ is a substituted or unsubstituted benzyl moiety. In other embodiments, R₂ is a substituted benzyl moiety. In yet other embodiments, R₂ is a substituted benzyl moiety, substituted with at least one hydroxyl group.

In certain embodiments, R₂ is

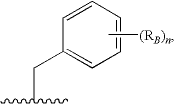

wherein each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and n is an integer between 1 and 5, inclusive. In certain embodiments, R₂ is

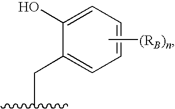

wherein each occurrence of R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and n is an integer between 1 and 5, inclusive. In certain embodiments, R₂ is

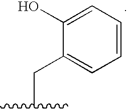

In other embodiments, R₂ is

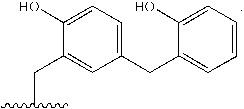

In yet other embodiments, R₂ is

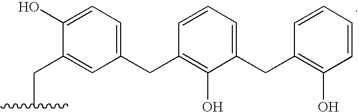

In certain embodiments, R₂ is a substituted or unsubstituted, branched or unbranched cyclicaliphatic moiety. In certain embodiments, R₂ is a substituted or unsubstituted, branched or unbranched cyclicalkyl moiety. In certain embodiments, R₂ is a substituted or unsubstituted, branched or unbranched cyclicalkenyl moiety. In certain embodiments, R₂ is a substituted or unsubstituted, branched or unbranched cyclicalkynyl moiety. In other embodiments, R₂ is a substituted or unsubstituted, branched or unbranched heterocyclicaliphatic moeity. In certain embodiments, R₂ is a substituted or unsubstituted, branched or unbranched heterocyclicalkyl moiety. In certain embodiments, R₂ is a substituted or unsubstituted, branched or unbranched heterocyclicalkenyl moiety. In certain embodiments, R₂ is a substituted or unsubstituted, branched or unbranched heterocylicalkynyl moiety. In certain embodiments, R₂ comprises a monocyclic ring system. In certain specific embodiments, R₂ comprises a 5-7-membered monocyclic ring system. In other embodiments, R₂ comprises a bicyclic ring system. In certain embodiments, R₂ comprises a bicyclic ring system with 5-membered or 6-membered rings.

In certain embodiments, R₁ and R₂ are the same. In other embodiments, R₁ and R₂ are different.

In certain embodiments, R₃ is a substituted or unsubstituted, branched or unbranched aliphatic moiety. In other embodiments, R₃ is a substituted or unsubstituted alkyl moiety. In certain embodiments, R₃ is a substituted or unsubstituted C₁-C₆alkyl moiety. In certain particular embodiments, R₃ is methyl, ethyl, or propyl. In certain embodiments, R₃ is methyl. In yet other embodiments, R₃ is a substituted or unsubstituted alkenyl moiety. In other embodiments, R₃ is a substituted or unsubstituted alkynyl moiety. In certain embodiments, R₃ is a substituted or unsubstituted cyclic moeity. In certain embodiments, R₃ is a substituted or unsubstituted heterocyclic moeity. In certain embodiments, R₃ is a substituted or unsubstituted aryl moiety. In other embodiments, R₃ is a substituted or unsubstituted heteroaryl moiety. In certain embodiments, R₃ comprises a six-membered ring. In other embodiments, R₃ comprises a five-membered ring. In yet other embodiments, R₃ comprises a seven-membered ring. In certain embodiments, R₃ is a substituted or unsubstituted phenyl moiety. In other embodiments, R₃ is an unsubstituted phenyl moiety. In other embodiments, R₃ is a phenyl moiety substituted with at least one hydrxyol group. In certain embodiments, R₃ is a substituted or unsubstituted, branched or unbranched arylaliphatic, heteroarylaliphatic, cyclicaliphatic, or heterocyclic aliphatic moiety. In certain embodiments, R₃ is benzyl. In certain embodiments, the carbon with the R₃ substituent is in the S-configuration. In other embodiments, the carbon with the R₃ substituent is in the R-configuration.

In certain embodiments, x is 2, and both R₃ are C₁-C₆alkyl. In certain embodiments, x is 2, and both R₃ are methyl, ethyl, or propyl. In certain embodiments, x is 2, and both R₃ are methyl. In certain embodiments, when x is 2, one R₃ is C₁-C₆alkyl, and the other R₃ is hydrogen. In certain embodiments, when x is 2, one R₃ is methyl, and the other R₃ is hydrogen.

In certain embodiments, R₄ is a substituted or unsubstituted, branched or unbranched aliphatic moiety. In other embodiments, R₄ is a substituted or unsubstituted alkyl moiety. In certain embodiments, R₄ is a substituted or unsubstituted C₁-C₆alkyl moiety. In certain particular embodiments, R₄ is methyl, ethyl, or propyl. In certain embodiments, R₄ is methyl. In yet other embodiments, R₄ is a substituted or unsubstituted alkenyl moiety. In other embodiments, R₄ is a substituted or unsubstituted alkynyl moiety. In certain embodiments, R₄ is a substituted or unsubstituted cyclic moeity. In certain embodiments, R₄ is a substituted or unsubstituted heterocyclic moeity. In certain embodiments, R₄ is a substituted or unsubstituted aryl moiety. In other embodiments, R₄ is a substituted or unsubstituted heteroaryl moiety. In certain embodiments, R₄ comprises a six-membered ring. In other embodiments, R₄ comprises a five-membered ring. In yet other embodiments, R₄ comprises a seven-membered ring. In certain embodiments, R₄ is a substituted or unsubstituted phenyl moiety. In other embodiments, R₄ is an unsubstituted phenyl moiety. In other embodiments, R₄ is a phenyl moiety substituted with at least one hydrxyol group. In certain embodiments, R₄ is a substituted or unsubstituted, branched or unbranched arylaliphatic, heteroarylaliphaic, cyclicaliphatic, or heterocyclic aliphatic moiety. In certain embodiments, R₄ is benzyl. In certain embodiments, the carbon with the R₄ substituent is in the S-configuration. In other embodiments, the carbon with the R₄ substituent is in the R-configuration. In certain embodiments, R₄ is hydrogen. In other embodiments, R₄ is a halogen. In certain embodiments, R₄ is fluorine. In other embodiments, R₄ is substituted or unsubstituted, branched or unbranched aliphatic moiety. In other embodiments, R₄ is a substituted or unsubstituted alkyl moiety. In other embodiments, R₄ is hydroxy or alkoxy. In other embodiments, R₄ is amino. In certain embodiments, y is 2, and both R₄ are hydrogen.

In certain embodiments, x is 1. In other embodiments, x is 2. When the dashed line is a carbon-carbon bond, then x is 1.

In certain embodiments, y is 1. In other embodiments, y is 2. When the dashed line is a carbon-carbon bond, then y is 1.

In certain embodiments, the dashed line represents a carbon-carbon bond, thereby resulting in a carbon-carbon double bond in the compound. In other embodiments, the dashed line does not represent a bond, thereby resulting in just a carbon-carbon single bond in the compound.

In certain embodiments, the compound is of the formula:

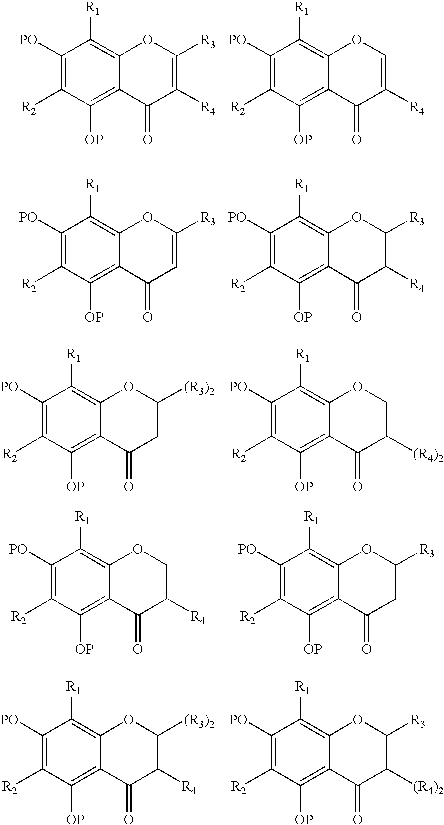

wherein R₁, R₂, R₃, R₄, and P are defined as above.

In other embodiments, the compound is of the formula:

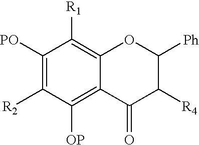

wherein R₁, R₂, R₄, and P are defined as above; and

Ph is a substituted or unsubstituted phenyl moiety.

In other embodiments, the compound is of the formula:

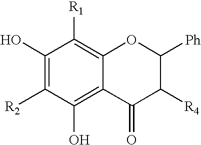

wherein R₁, R₂, R₄, and P are defined as above; and

Ph is a substituted or unsubstituted phenyl moiety.

In certain embodiments, the compound is of the formula:

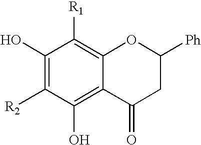

wherein R₁ and R₂ are defined as above; and Ph is a substituted or unsubstituted phenyl moeity. In certain embodiments, the phenyl moiety is unsubstituted. In other embodiments, the phenyl is substituted with at least one hydroxyl moiety. In other embodiments, the compound has the stereochemistry depicted in the formula:

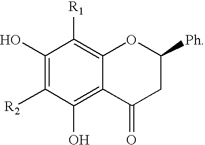

In yet other embodiments, the compound has the stereochemistry depicted in the formula:

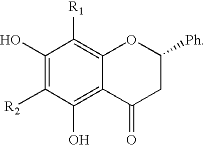

In other embodiments, the compound is of the formula:

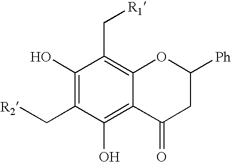

wherein

R₁′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —CN; —SCN; —SRA; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —CN; —SCN; —SR_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

Ph is a substituted or unsubstituted phenyl moiety. In certain embodiments, Ph is an unsubstituted phenyl moiety. In other embodiments, Ph is a phenyl moiety substituted with at least on hydroxyl group. In certain embodiments, the compound has the stereochemistry depicted in the formula:

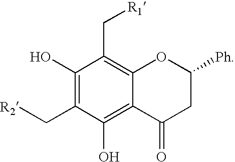

In other embodiments, the compounds has the stereochemistry depicted in the formula:

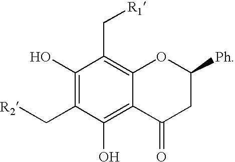

In other embodiments, the compound is of the formula:

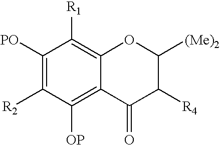

wherein R₁, R₂, R₄, and P are defined as above.

In other embodiments, the compound is of the formula:

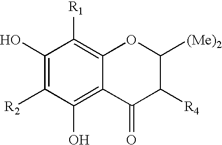

wherein R₁, R₂, R₄, and P are defined as above.

In certain embodiments, the compound is of the formula:

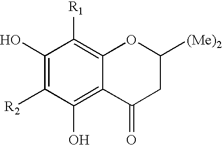

wherein R₁ and R₂ are defined as above.

In other embodiments, the compound is of the formula:

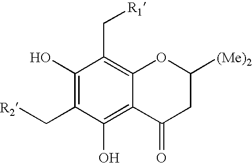

wherein

R₁′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —CN; —SCN; —SRA; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —CN; —SCN; —SR_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, R₁′ and R₂′ are each independently a substituted or unsubstituted, monocyclic, bicyclic, or tricyclic ring system. In other embodiments, R₁′ and R₂′ are each independently a substituted or unsubstituted, monocyclic or bicyclic ring system. In still other embodiments, R₁′ and R₂′ are each independently a substituted or unsubstituted, monocyclic ring system. The monocyclic ring system is five-membered in certain embodiments and six-membered in other embodiments. In yet other embodiments, R₁′ and R₂′ are each independently a substituted or unsubstituted, bicyclic ring system. The bicyclic ring system is two fused six-membered rings in certain embodiments. In other embodiments, the bicyclic ring system is a six-membered ring fused to a five-membered ring; or a five-membered ring fused to another five-membered ring.

In certain embodiments, R₁′ and R₂′ are each independently a substituted or unsubstituted aryl moiety. In certain particular embodiments, R₁′ and R₂′ are each independently a substituted aryl moiety. In certain embodiments, at least one of R₁′ and R₂′ is a substituted aryl moiety with at least one hydroxyl substituent. In other embodiments, R₁′ and R₂′ are each independently a substituted or unsubstituted heteroaryl moiety. In still further embodiments, R₁′ and R₂′ are each independently a substituted heteroaryl moiety. In yet other embodiments, at least one of R₁′ and R₂′ is a substituted heteroaryl moiety with at least one hydroxyl substituent.

In certain embodiments, R₁′ is selected from the group consisting of:

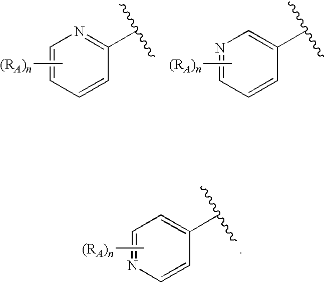

In other embodiments, R₁′ is selected from the group consisting of:

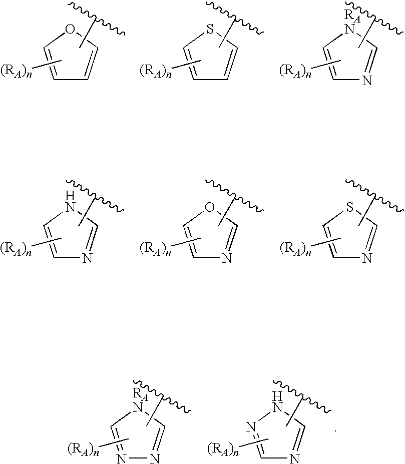

In other embodiments, R₂′ is selected from the group consisting of:

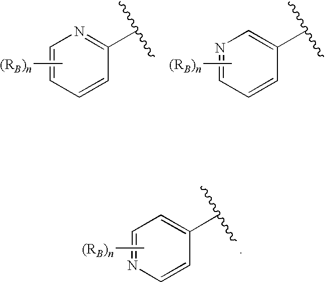

In other embodiments, R₂′ is selected from the group consisting of:

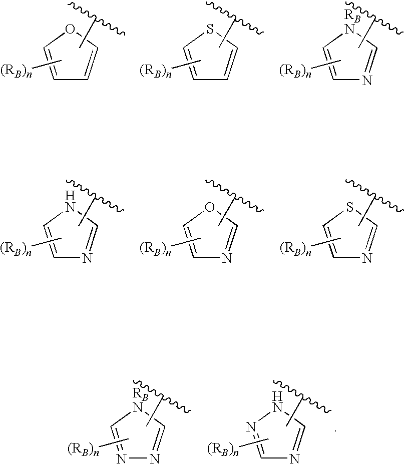

In certain embodiments, R₁′ and R₂′ are substituted or unsubstituted, six-membered, heterocyclic or carbocyclic moieties. Exemplary compounds of this embodiment are of the formula:

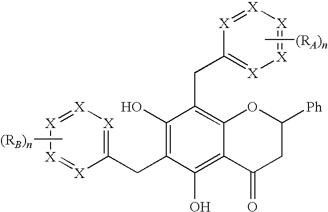

wherein

X is C or N and there are no more than three N atoms in any ring;

each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of n is an integer between 1 and 5, inclusive; and

Ph is a substituted or unsubstituted phenyl moiety. In certain embodiments, only one X per ring is N, the other Xs being C. In other embodiments, at least one X per ring is a N, the balance being C. Other exemplary compounds of this embodiment are of the formula:

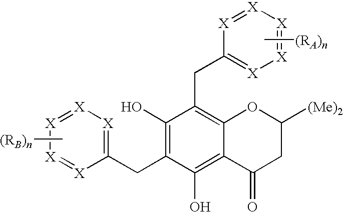

wherein

X is C or N and there are no more than three N atoms in any ring;

each occurrence of R_{A} is independently halogen; cyclic or acyclic, substituted or unsubstituted, branched or unbranched aliphatic; cyclic or acyclic, substituted or umsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently halogen; cyclic or acyclic, substituted or unsubstituted, branched or unbranched aliphatic; cyclic or acyclic, substituted or unsubstituted, branched or umbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

each occurrence of n is an integer between 1 and 5, inclusive. In certain embodiments, only one X per ring is N, the other Xs being C. In other embodiments, at least one X per ring is a N, the balance being C.

In certain embodiments, the compound is of the formula:

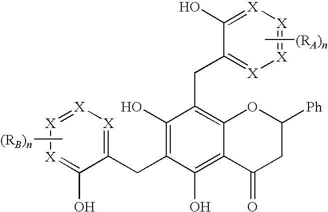

wherein X, R_{A}, R_{B}, Ph, and n are defined as above.

In other embodiments, all occurrences of X are carbon. Compounds of this embodiment are of the formula:

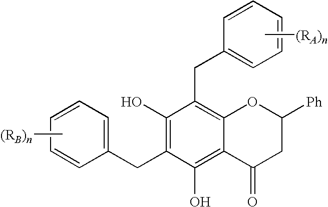

wherein

X is C or N and there are no more than three N atoms in any ring;

each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, allylamino, dialkylamino, heteroaryloxy; or heteroarylthio moiety;

each occurrence of R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of n is an integer between 1 and 5, inclusive; and

Ph is a substituted or unsubstituted phenyl moiety.

In certain particular embodiments, the compound is of the formula:

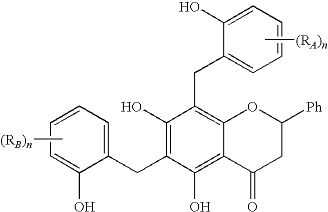

wherein R_{A}, R_{B}, Ph, and n are defined as above.

In other embodiments, the compound is of the formula:

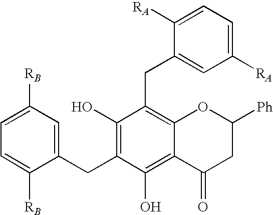

wherein R_{A}, R_{B}, and Ph are defined as above.

In other embodiments, the compound is of the formula:

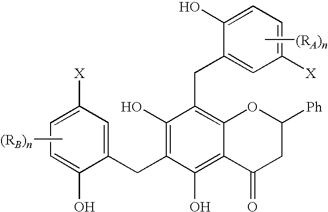

wherein

R_{A}, R_{B}, Ph, and n are defined as above; and

each occurrence of X is independently fluorine, chlorine, bromine, or iodine. In certain embodiments, both Xs are chlorine. In other embodiments, X is fluorine, chlorine, or bromine. In yet other embodiments, X is fluorine or chlorine. In certain embodiments, Ph is an unsubstituted phenyl moiety. In other embodiments, Ph is a substituted phenyl moiety. In yet other embodiments, Ph is a phenyl moiety substituted with a hydroxyl group.

Exemplary compounds of the invention include:

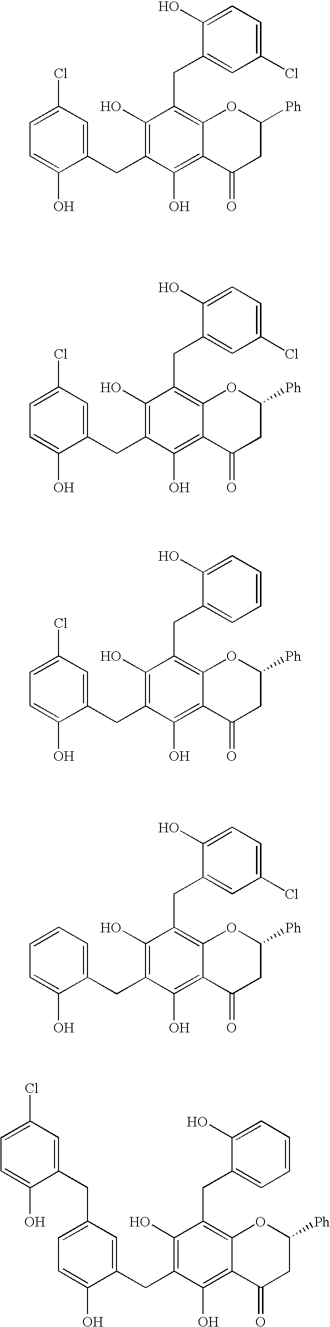

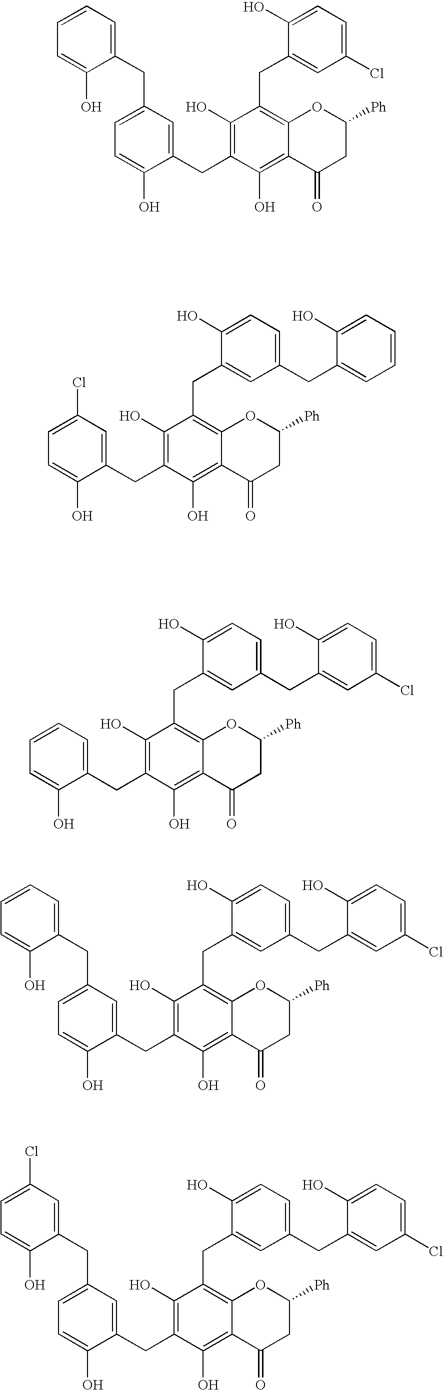

In certain embodiments, the compound is of the formula:

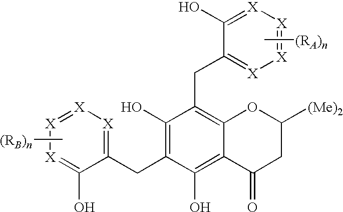

wherein X, R_{A}, R_{B}, and n are defined as above.

In other embodiments, all occurrences of X are carbon. Compounds of this embodiment are of the formula:

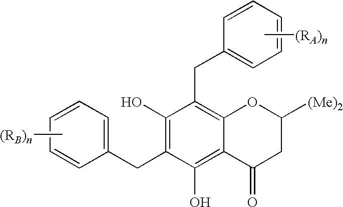

wherein

X is C or N and there are no more than three N atoms in any ring;

each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{B} is independently halogen; cyclic or acyclic, substituted or unsubstituted, branched or umbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of n is an integer between 1 and 5, inclusive; and

Ph is a substituted or unsubstituted phenyl moiety.

In certain particular embodiments, the compound is of the formula:

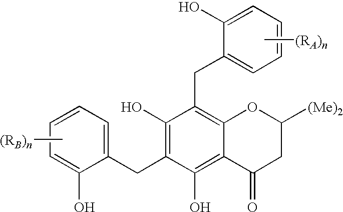

wherein R_{A}, R_{B}, Ph, and n are defined as above.

In other embodiments, the compound is of the formula:

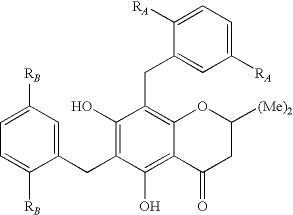

wherein R_{A}, R_{B}, and Ph are defined as above.

In other embodiments, the compound is of the formula:

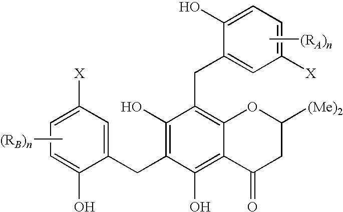

wherein

R_{A}, R_{B}, Ph, and n are defined as above; and

each occurrence of X is independently fluorine, chlorine, bromine, or iodine. In certain embodiments, both Xs are chlorine. In other embodiments, X is fluorine, chlorine, or bromine. In yet other embodiments, X is fluorine or chlorine. In certain embodiments, Ph is an unsubstituted phenyl moiety. In other embodiments, Ph is a substituted phenyl moiety. In yet other embodiments, Ph is a phenyl moiety substituted with a hydroxyl group.

Exemplary compounds of the invention include:

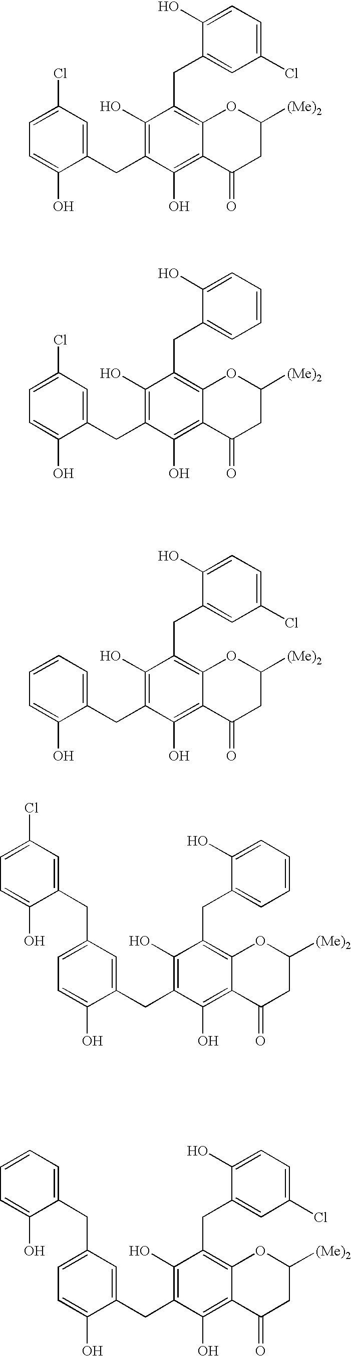

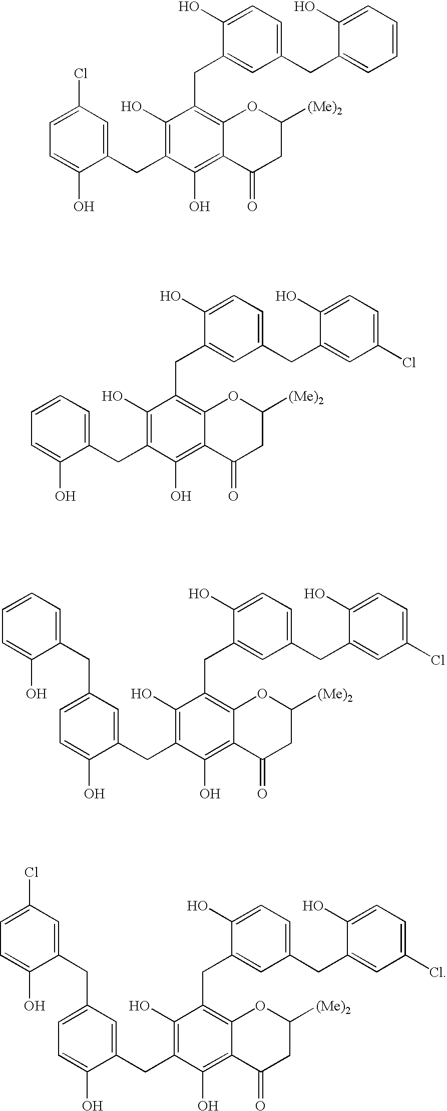

### Methods of Synthesis

The present invention also includes all steps and methodologies used in preparing the compounds of the invention as well as intermediates along the synthetic route. The present invention provides for the modular synthesis of polyphenols by modifying a core structure. The core structure is pinocembrin, chrysin, or other derivative thereof. This methodology allows for the addition of the same substituents to the core structure, or for the addition of two different substituents to the core structure. As described below, that is, Ar₂ and Ar₃ may be the same or different.

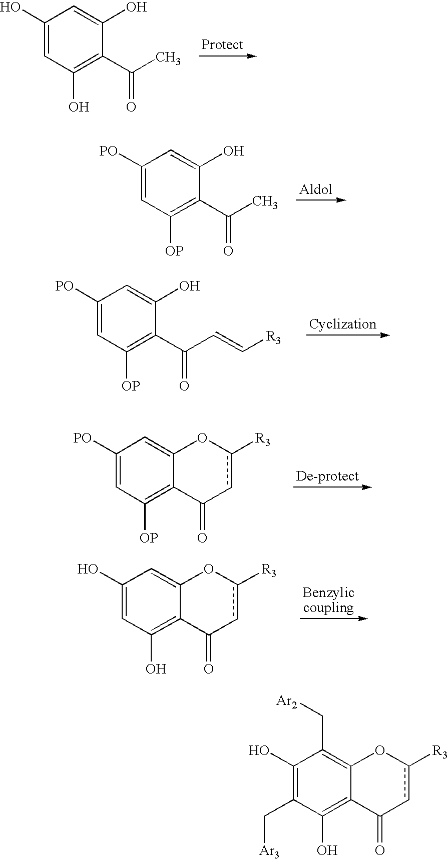

The synthesis begins with the preparation of the core structure, pinocembrin or chrysin. Trihydroxyacetophenone is selectively bis-protected. The protected-acetophenone is then converted to chalcone via an Aldol condensation. In certain embodiments, benzaldehyde is used in the Aldol condensation reaction. A derivative of benzaldehyde may also be used. Cyclization is then effected under basic conditions to yield a protected form of pinochembrin. The protecting groups are then removed to yield pinocembrin. Pinocembrin or a protected form or derivative thereof may be oxidized to yield chrysin of a derivative thereof.

In one embodiment, pinocembrin is prepared by the steps of:
(a) selectively bis-protecting 2′,4′,6′-trihydroxyaceptophenone to yield 2′,4′-bis(protected)-6′-hydroxyacetophenone;
(b) condensing 2′,4′-bis(protected)-6′-hydroxyacetophenone with an aldehyde (e.g., benzaldehyde or a derivative thereof) or ketone;
(c) cyclizing 2′,4′-bis(protected)-6′-hydroxychalcone; and
(d) optionally, deprotecting (±)-2′,4′-bis(protected)flavanone to produce (±)-pinocembrin. In certain embodiments, 2′,4′,6′-trihydroxyaceptophenone is protected using methylchloromethyl ether; therefore, the protecting group is MOM. The step of protecting is performed under basic conditions, e.g., in the presence of an amine (e.g., diisopropylethylamine). The step of condensing is typically an Aldol reaction. The Aldol reaction is performed under basic conditions. Any aldehyde or ketone may be used. In certain embodiments, a ketone is used. In other embodiments, an aldehyde is used. In certain embodiments, benzaldehyde is used. Besides benzaldehyde, other derivatives of benzaldehyde may also be used. For example, the phenyl ring of benzaldehyde may be substituted. The step of cyclizing is performed under basic conditions. In certain embodiments, the step of cyclizing is performed in the presence of NaOAc. In certain embodiments, when the MOM-protecting group is used, the step of deprotecting is performed in the presence of an acid (e.g., HCl).

The pinocembrin or chrysin core structure is then functionalized to yield the inventive compounds. In certain embodiments, compounds of the formula:

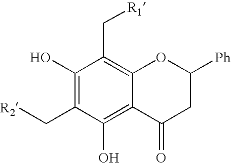

wherein

R₁′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —CN; —SCN; —SRA; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, diallcylamino, heteroaryloxy; or heteroarylthio moiety;

R₂′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or uibranched aryl; substituted or unsubstituted, branched or unbranched heteroaryl; —OR_{B}; —CN; —SCN; —SR_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

Ph is a substituted or unsubstituted phenyl moiety; are prepared by the method comprising:

benzylating pinocembrin using a benzyl alcohol of formula:

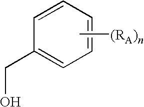

wherein

each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

n is an integer between 1 and 5, inclusive.

In certain embodiments, the step of benzylating is performed in the presence of a Lewis acid. Lewis acids useful in the present methodology include, but are not limited to, Sc(OTf)₃, AlCl₃, SnCl₄, Mg(OTf)₂, ZnCl₂, ZnBr₂, and ZnI₂. In certain particular embodiments, ZnCl₂ is used as the Lewis acid. In certain embodiments, stoichiometric amounts of the Lewis acid are used (e.g., 0.5 eqiv. 1 equiv., 1.5 equiv., 2 equiv., 2.5 equiv., 3 equiv., etc.). In other embodiments, catalytic amounts of the Lewis acid are used. In other embodiments, the step of benzylating is performed in the presence of a protic acid (e.g., p-TsOH, CSA, etc.). The benzylation reaction is performed at a temperature ranging from 20° C. to 150° C. In certain embodiments, the reaction is performed at a temperature ranging from 60° C. to 130° C. In certain particular embodiments, the reaction is run at approximately 90° C., 95° C., 100° C., 105° C., 110° C., 115° C., 120° C., 125° C., 130° C., 135° C., 140° C., 145° C., or 150° C. In certain embodiments, the benzylic coupling reaction is performed with the irradiation of the reaction mixture with microwaves. The microwave reaction is typically performed at approximately 130° C. The microwave reactions may be performed at temperatures ranging from 100° C. to 160° C., or from 120° C. to 140° C. The reaction is performed in any organic solvent. In certain embodiments, the solvent is aprotic. Dioxane is particularly useful as a solvent in the benzylation reaction. The benzylation reaction time is approximately 0.5, 1, 1.5, 2, 3, 4, 5, 6, 7, 8, 9, 10, 11, 12, 15, 24, or 48 hours.

In certain embodiments, the benzyl alcohol used in the benzylation reaction is of the formula:

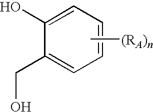

wherein R_{A} and n are as defined above. In certain embodiments, the benzyl alcohol is of the formula:

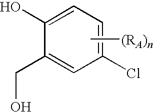

wherein R_{A} and n are as defined above. In certain other embodiments, the benzyl alcohol is of the formula:

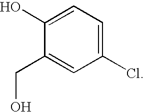

In other embodiments, the benzyl alcohol is of the formula:

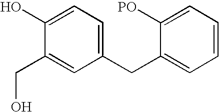

wherein P is hydrogen or an oxygen protecting group. In certain embodiments, P is a silyl-protecting group. In certain particular embodiments, P is TBDPS.

The benzyl alcohols described above are also considered part of the invention.

Other intermediates that are useful in the synthesis of the inventive compounds and are considered part of the invention include pinocembrin and chrysin derivatives. In certain embodiments, the intermediates are of the formula:

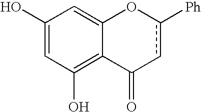

wherein Ph is a substituted or unsubstituted phenyl moiety; and
the dashed line represents the presence of absence of a bond. In certain embodiments, the Ph moiety is substituted. In other embodiments, the Ph moiety is unsubstituted. In certain embodiments, the intermediate has the stereochemistry as shown:

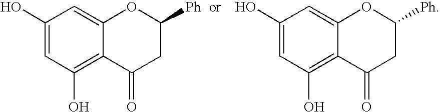

In certain embodiments, chrysin and pinocembrin are not included.

Various exemplary reactions used in the syntheses of compounds of the invention are shown in the figures and are described in the Examples section below. As will be appreciated by one of skill in the art, various isolation and purification techniques including flash chromatography, crystallization, distillation, HPLC, thin layer chromatography, extraction, filtration, etc. may be used in the course of synthesizing compounds of the invention. These techniques may be used in the preparation or purification of intermediates, reagents, products, starting materials, or solvents.

### Pharmaceutical Compositions

This invention also provides a pharmaceutical preparation comprising at least one of the compounds as described above and herein, or a pharmaceutically acceptable derivative thereof, which compounds inhibit the growth of or kill microorganisms, and, in certain embodiments of special interest are inhibit the growth of or kill antibiotic-resistant organisms including methicillin-resistant organisms, vancomycin-resistant organisms, and penicillin-resistant organisms. In other embodiments, the compounds show cytostatic or cytotoxic activity against neoplastic cells such as cancer cells. In yet other embodiments, the compounds inhibit the growth of or kill rapidly dividing cells such as stimulated inflammatory cells.

As discussed above, the present invention provides novel compounds having antimicrobial and antiproliferative activity, and thus the inventive compounds are useful for the treatment of a variety of medical conditions including infectious diseases, cancer, autoimmune diseases, inflammatory diseases, and diabetic retinopathy. Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents, e.g., another anti-microbial agent or another anti-proliferative agent. In other embodiments, these compositions further comprise an anti-inflammatory agent such as aspirin, ibuprofen, acetaminophen, etc., pain reliever, or anti-pyretic.

It will also be appreciated that certain of the compounds of the present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19, 1977; incorporated herein by reference. The salts can be prepared in situ during the final isolation and purification of the compounds of the invention, or separately by reacting the free base functionality with a suitable organic or inorganic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates. In certain embodiments, the esters are cleaved by enzymes such as esterases.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicles, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Uses of Compounds and Pharmaceutical Compositions

The invention further provides a method of treating infections and inhibiting tumor growth. The method involves the administration of a therapeutically effective amount of the compound or a pharmaceutically acceptable derivative thereof to a subject (including, but not limited to a human or animal) in need thereof.

The compounds and pharmaceutical compositions of the present invention may be used in treating or preventing any disease or conditions including infections (e.g., skin infections, GI infection, urinary tract infections, genito-urinary infections, systemic infections), proliferative diseases (e.g., cancer), and autoimmune diseases (e.g., rheumatoid arthritis, lupus). The compounds and pharmaceutical compositions may be administered to animals, preferably mammals (e.g., domesticated animals, cats, dogs, mice, rats), and more preferably humans. Any method of administration may be used to deliver the compound of pharmaceutical compositions to the animal. In certain embodiments, the compound or pharmaceutical composition is administered orally. In other embodiments, the compound or pharmaceutical composition is administered parenterally.

In yet another aspect, according to the methods of treatment of the present invention, bacteria are killed, or their growth is inhibited by contacting the bacteria with an inventive compound or composition, as described herein. Thus, in still another aspect of the invention, a method for the treatment of infection is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for killing or inhibiting the growth of bacteria. The compounds and compositions, according to the method of the present invention, may be administered using any amount and any route of administration effective for killing or inhibiting the growth of bacteria. The exact amount required will vary from subject to subject, depending on the species, age, and general condition of the subject, the severity of the infection, the particular compound, its mode of administration, its mode of activity, and the like. The compounds of the invention are preferably formulated in dosage unit form for ease of administration and uniformity of dosage. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such an Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creanis, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

### Screening of Inventive Compounds

The compounds described herein may be screened for any biological activity. In certain embodiments, the compounds are screened using known assays in the art. In certain embodiments, the compounds are screened for anti-microbial activity. For example, assays may be used to determined the concentration of the compound necessary to inhibit microbial growth by 50%. In certain embodiments, the compounds are tested against bacteria. The compounds may be tested against antibiotic-resistant bacteria (e.g., vancomycin-resistant bacteria, methicillin-resistant bacteria, penicillin-resistant bacteria, tetracycline-resistant bacteria, etc.). The compounds may be test in against microorganisms such as mycobacteria, fungi, yeast, protozoa, etc.

In other embodiments, the compounds are tested for their anti-neoplastic or anti-proliferative activity. Compounds with anti-neoplastic activity may be useful for treating diseases such as cancer, inflammation, auto-immune diseases, benign neoplasms, and diabetic retinopathy.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Synthesis of Antimicrobial Natural Products Targeting FtsZ: (+/−)-Dichamanetin and (+/−)-2′″-Hydroxy-5″-benzylisovarinol-B

The structural similarity between polyphenolic compounds 1-3 suggested that they might all derive their antimicrobial activity by inhibiting the GTPase activity of FtsZ. In order to test this hypothesis, we undertook the syntheses of compounds 2 and 3. While naturally-occurring flavanones have attracted the attention of synthetic chemists and biologists alike, benzylated flavanones are quite rare, and as such no efficient syntheses of compounds related to 2 and 3 have been reported (The synthesis of gericudranin, a related para-hydroxybenzylated 3-hydroxy flavanone, has been reported: Choi et al. Heterocycles 1996, 43, 1223-1228; incorporated herein by reference). A straightforward synthesis would allow us to evaluate the origin of their biological activity and prepare analogs that may be more potent.

TABLE 1
Antimicrobial activities (MICs, μM) of compounds 1-3

| | S. | B. | | | |
|---|---|---|---|---|---|
| compound | aureus | subtilis | M. smegmatis | E. coli | P. aeruginosa |
| 1 | 2.5 | 1.25 | —^{a} | 20 | 40 |
| 2 | 1.7 | 1.7 | 3.4 | —^{b} | —^{b} |
| 3 | 10.7 | 2.6 | 3.8 | 2.3 | 15.4 |

^{a}not evaluated.
^{b}no significant activity.

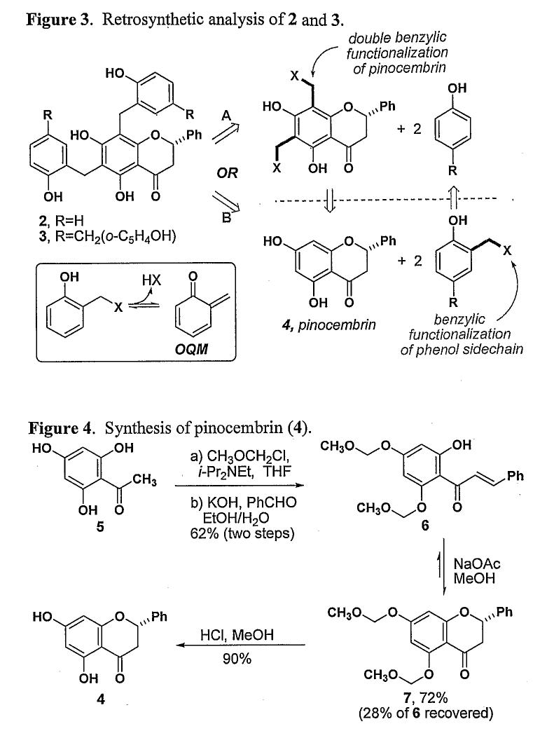

The substituent symmetry of 2 and 3 suggested that a common core could be elaborated to provide both molecules. The formation of benzylic carbon-carbon bonds with electron-rich arenes is often achieved via ortho quinone methide (OQM) intermediates, which can be accessed by a variety of routes (Van De Water et al. Tetrahedron 2002, 58, 5367-5405; incorporated herein by reference). Pinocembrin (4) could be converted to the OQM precursor by benzylic functionalization (FIG. 3, path A). We initially planned to explore halomethylation, hydroxymethylation, and aminomethylation, since all of these processes take place under neutral or acidic conditions. While all of these processes are well-established for phenols, the analogous transformations using resorcinols are almost unknown (Tabatabai et al. Tetrahedron Lett. 1990, 31, 3295-3298; Tabatabai et al. Suprainol. Chem. 1994, 4, 147-152; each of which is incorporated herein by reference).

Furthermore, the base sensitivity of the flavanone would limit the conditions that could be employed for the formation of the OQM intermediate. An alternate synthetic approach would involve functionalization of the incoming phenolic side-chain (FIG. 3, path B).

Our synthesis began with the development of an efficient route to pinocembrin (FIG. 3). Flavanones related to pinocembrin have been prepared in high yield from the reaction of phenols with cinnamoyl chlorides through a Friedel-Crafts/cyclization sequence (Lee, J.-M.; Tseng, T.-H.; Lee, Y.-J. Synthesis 2001, 2247-2254; Solladie et al. Eur. J. Org. Chem. 1999, 2309-2314; Talapatra, B.; Deb, T.; Talapatra, S. Ind. J. Chem., Sect. B 1986, 25B, 1122-1125; Suresh, R. V.; Iyer, C. S. R.; Iyer, P. R.; Heterocycles 1986, 24, 1925-1930; each of which is incorporated herein by reference). Since this process is known to be low yielding for pinocembrin (Huzise, S.-I.; Tatsita, H. Chem. Ber. 1941, 74B, 275-278; incorporated herein by reference), we developed an aldol condensation/cyclization route that rapidly provides multi-gram quantities of pinocembrin (Huang et al. Synth. Commun. 1999, 29, 1383-1392; Zhao, L.; Li, Y. Org. Prep. Proced. Int. 1996, 28, 165-171; each of which is incorporated herein by reference). Trihydroxyacetophenone 5 is selectively bis-protected with methylchloromethyl ether, then converted to chalcone 6 under standard conditions. Cyclization with sodium acetate provided an equilibrium mixture of the cyclized product and chalcone starting material. Acidic hydrolysis of the MOM groups provided pinocembrin 4.

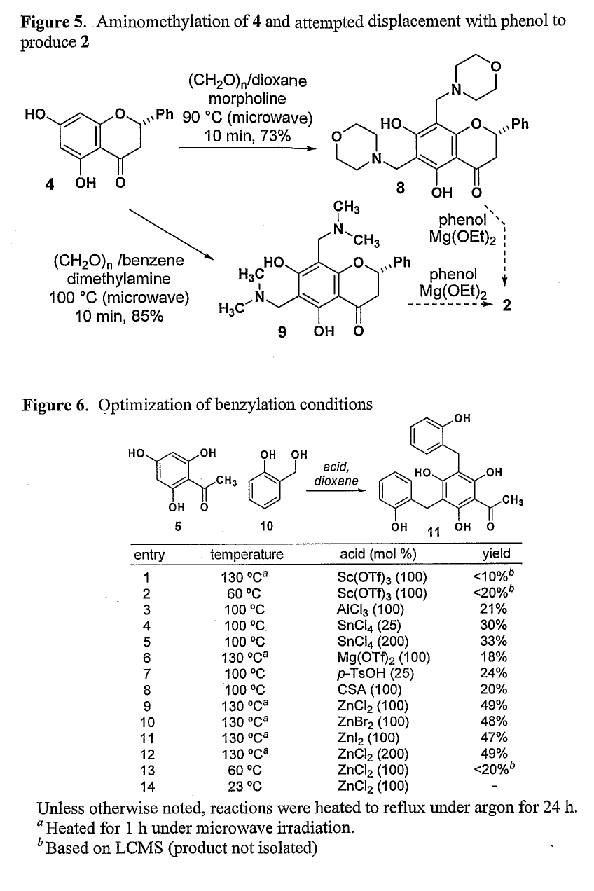

We explored several methods of benzylic functionalization of pinocembrin in an effort to prepare a suitable intermediate that would eventually lead to 2 and 3. We were able to produce both the morpholine (8, FIG. 5) and dimethylamine (9) Mannich bases from pinocembrin in high yield, though these reactions are not well established for complex resorcinol substrates (Ghantwal, S. R.; Samant, S. D. J. Indian Chem. Soc. 2000, 77, 100-101; Jerzmanowska, Z.; Jurkowska-Kowalczyk, E. Roczniki Chemii 1970, 44, 1395-1401; each of which is incorporated herein by reference). Additional experiments demonstrated that resorcinol-derived OQM intermediates were significantly more difficult to access than their phenol-derived counterparts.

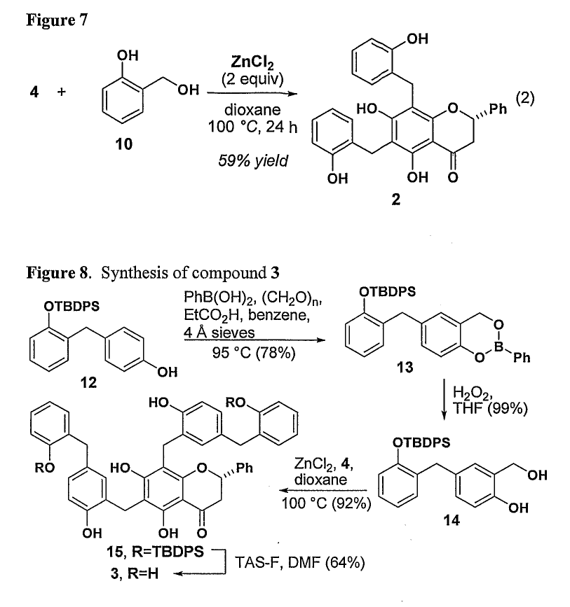

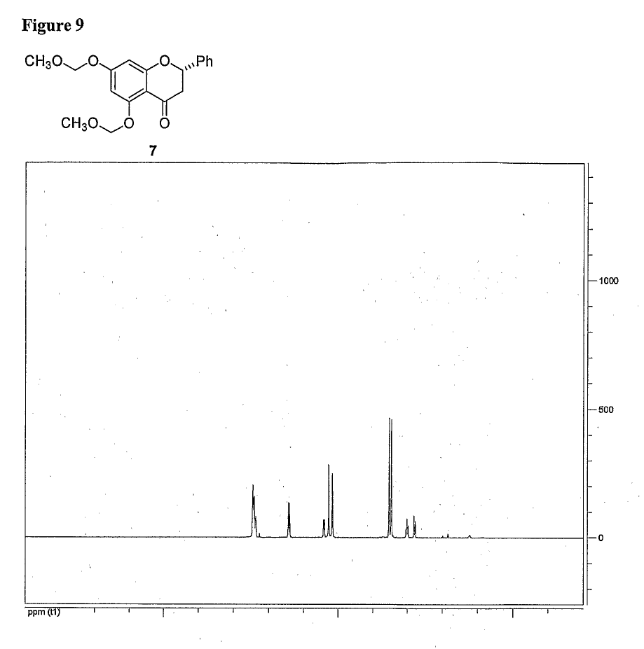

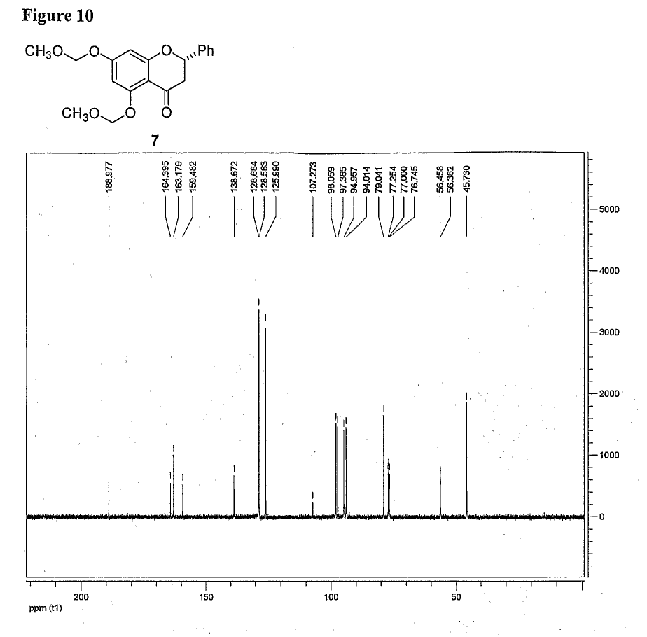

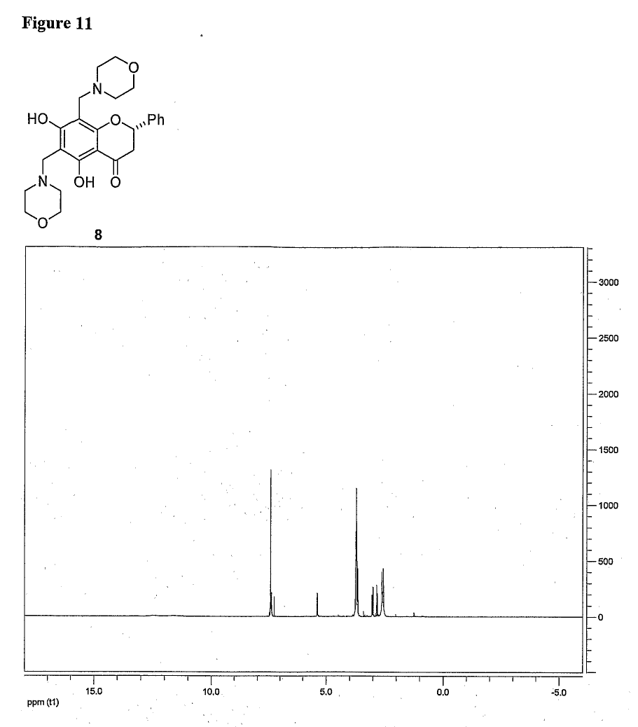

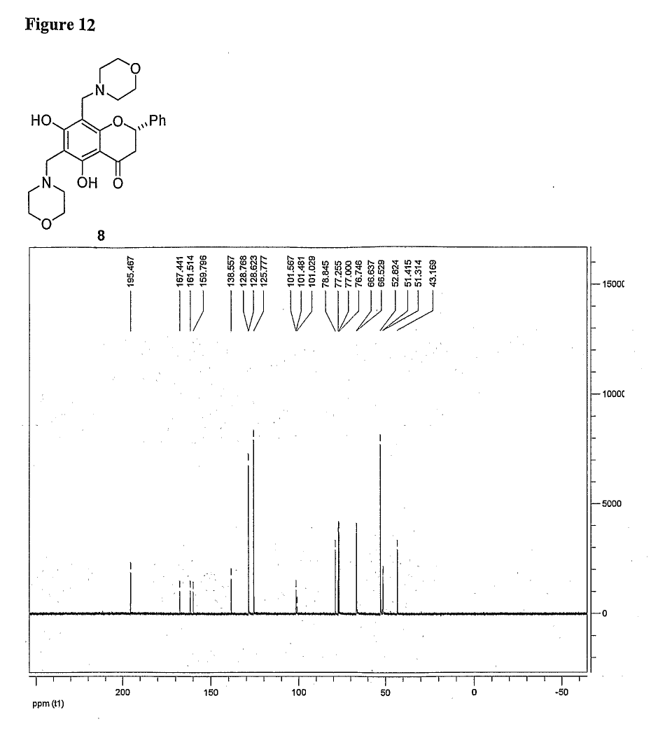

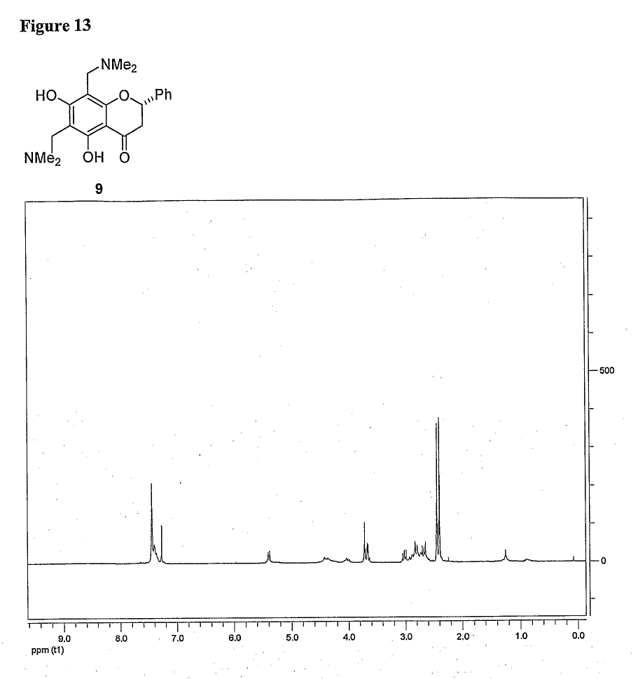

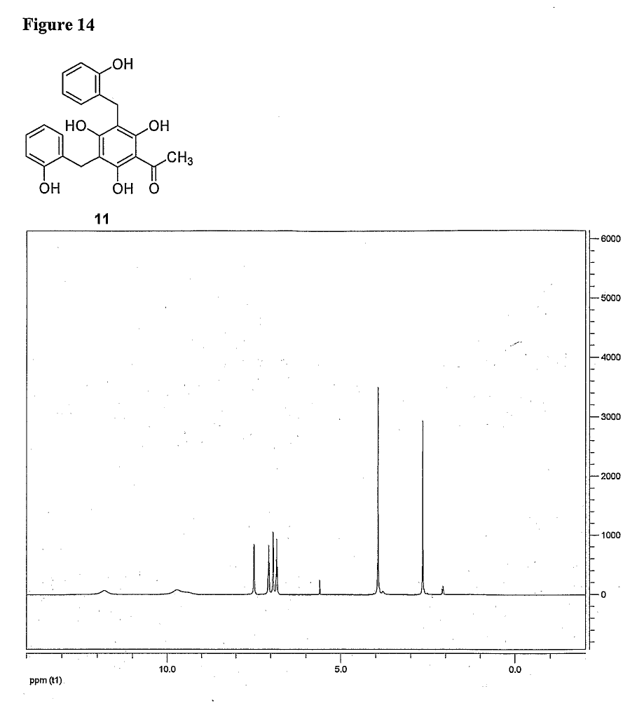

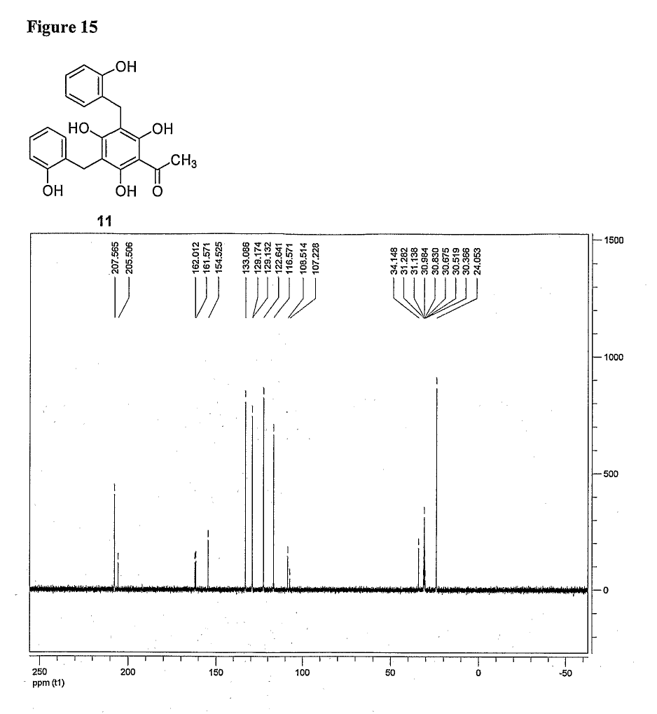

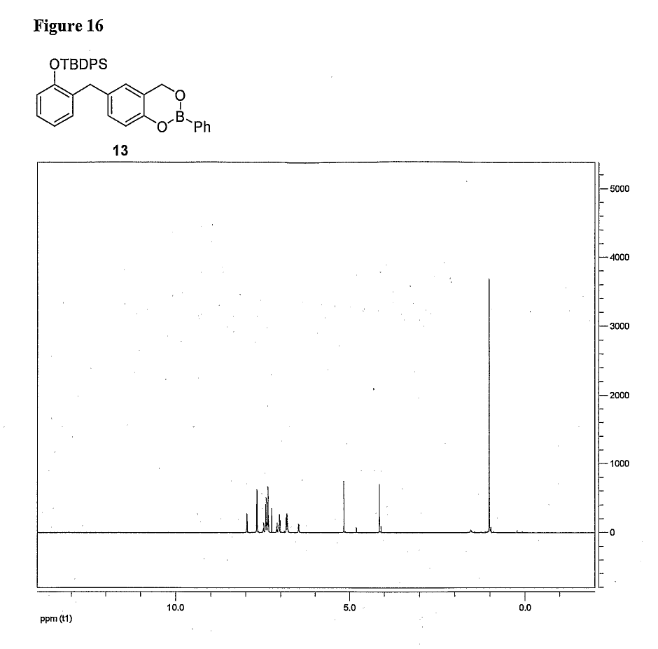

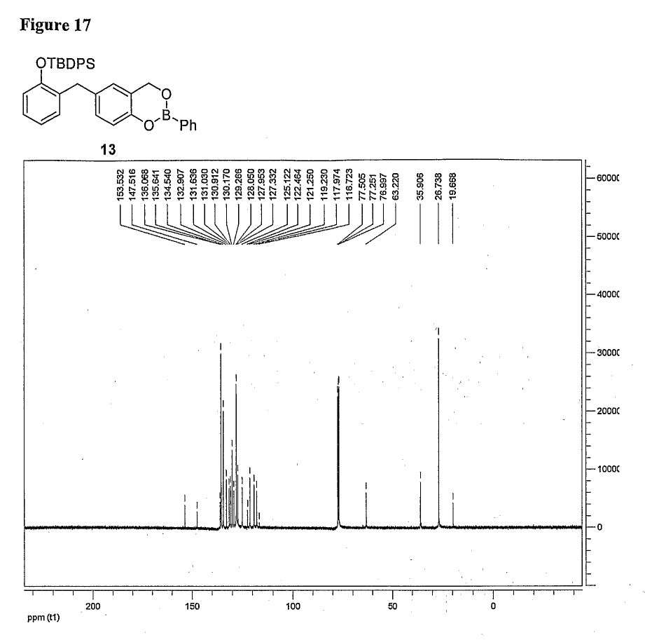

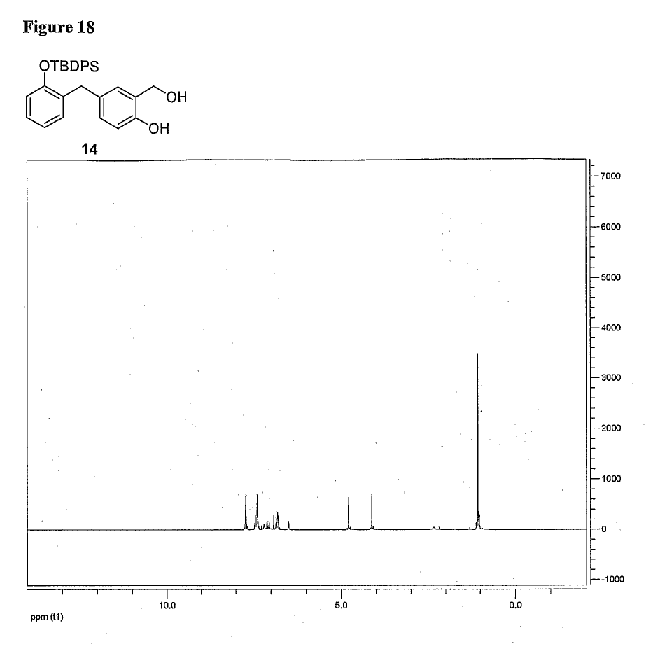

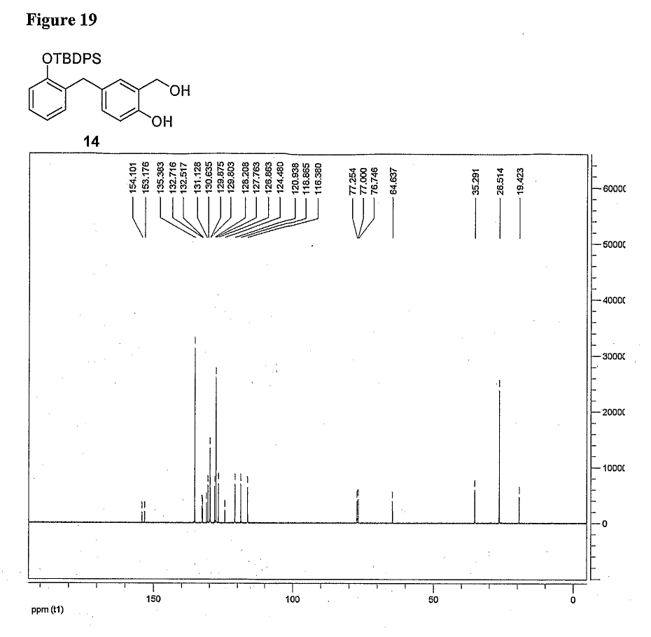

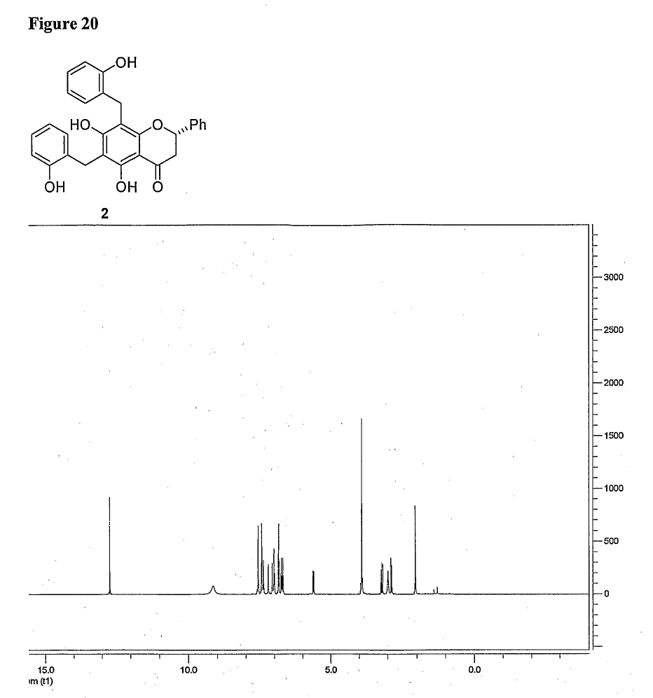

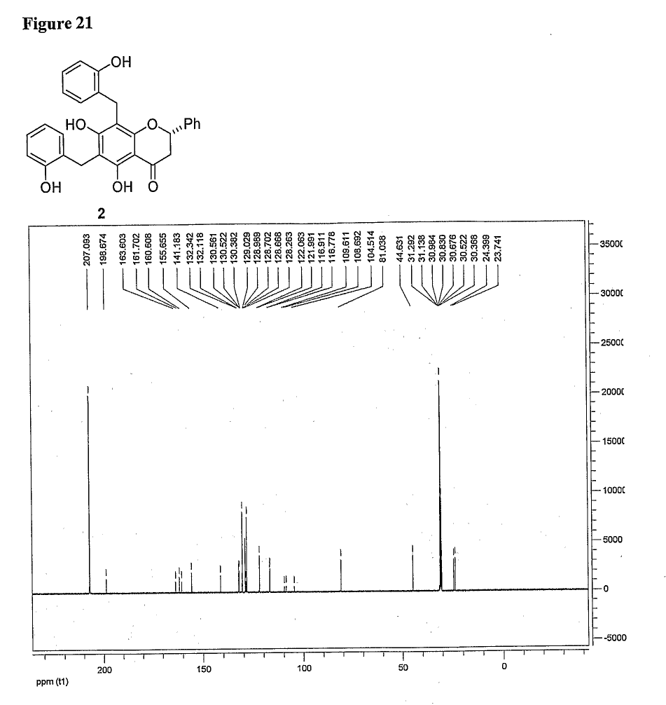

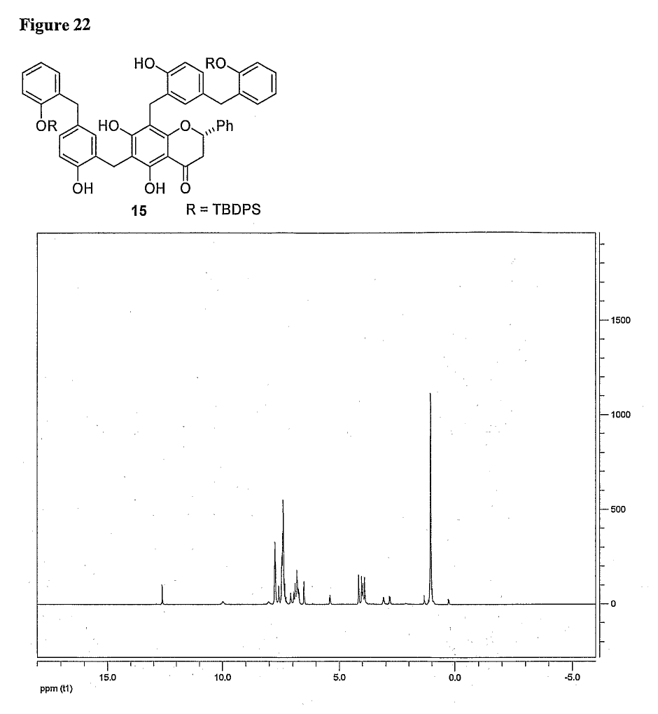

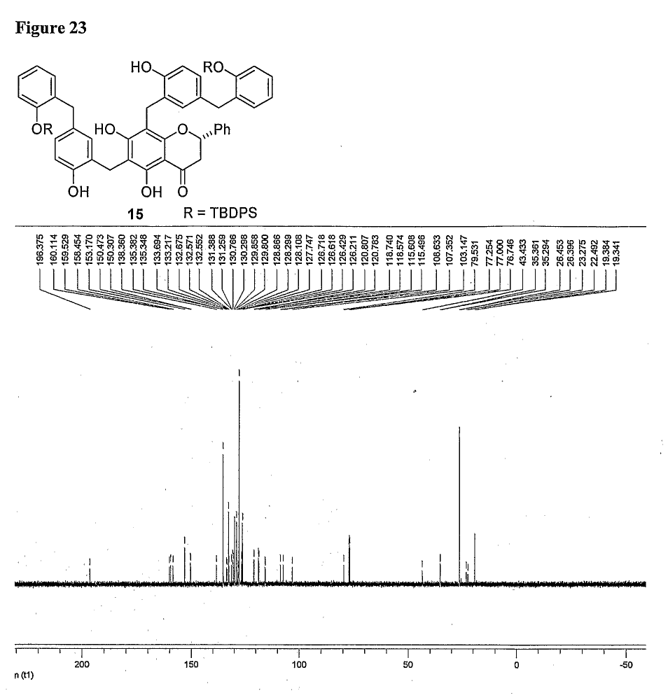

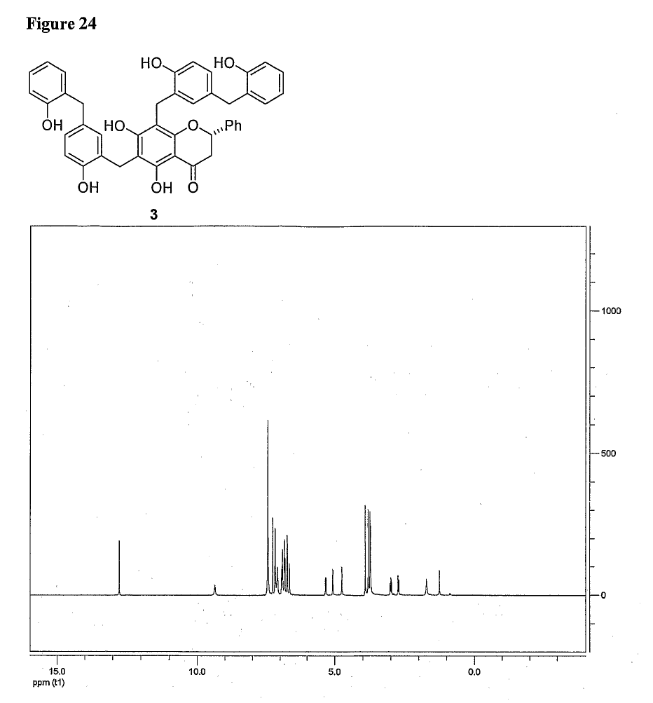

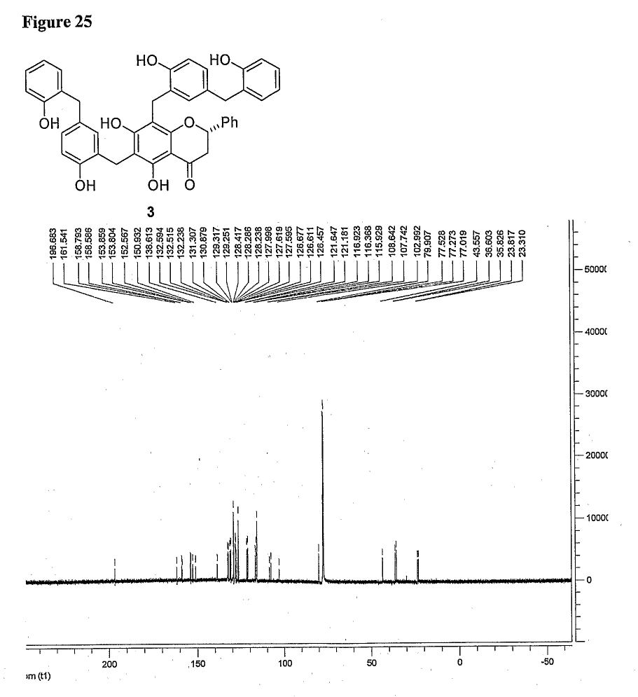

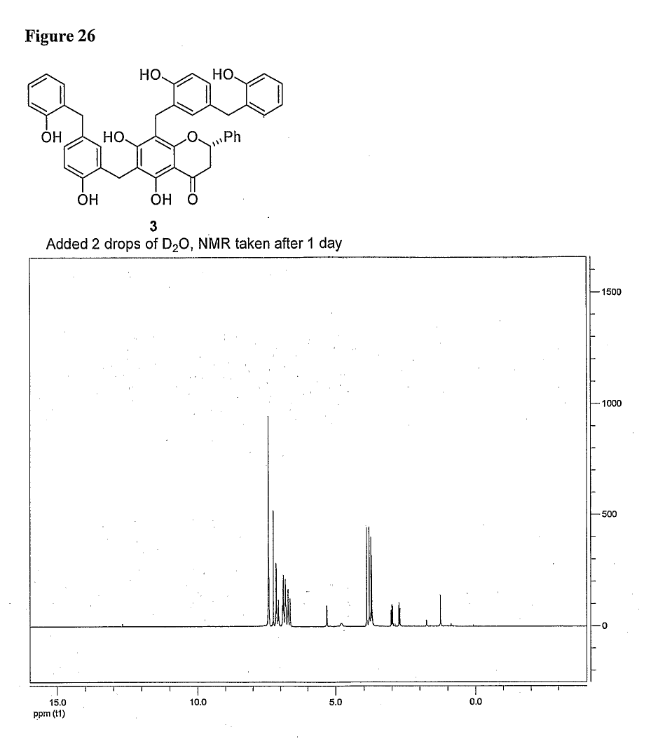

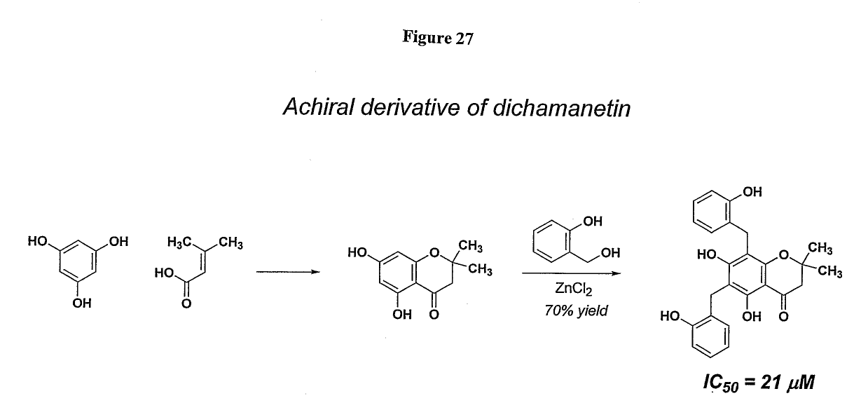

We turned our attention to the use of benzylic side-chain substrates (FIG. 3, path B). This process was known to be inefficient for the synthesis of dichamanetin (Dichamanetin was produced in <2% yield using BF₃—OEt₂ for this reaction: Lasswell, W. L., Jr.; Hufford, C. D. J. Org. Chem. 1977, 42, 1295-1302; incorporated herein by reference). We employed trihydroxyacetophenone 5 as a model system to investigate reaction conditions for the aryl alkylation reaction with o-hydroxybenzyl alcohol (FIG. 6). A survey of Lewis and protic acids revealed that ZnCl₂ in dioxane afforded bisalkylated product 11 in 49% yield (A related process has been reported using phenol-derived, but not resorcinol-derived, substrates. The reported reaction was conducted under conditions using a domestic microwave, and while the temperature was not monitored, the wattage applied suggests that the reaction takes place at >200° C.: IKhalafi-Nezhad, A.; Rad, M. N. S.; Hakimelahi, G. H. Helv. Chim. Acta 2003, 86, 2396-2403; incorporated herein by reference). Similar reaction conditions converted pinocembrin 4 to dichamanetin 2 in 59% yield to complete the first selective synthesis of this natural product (FIG. 7).

We next addressed the synthesis of 2′″-hydroxy-5″-benzyl-isouvarinol-B (3, FIG. 8). Phenol 12 is made by selective mono-protection of commercially available 2,4′-dihydroxydiphenylmethane (De Bruyn et al. Tetrahedron 1997, 53, 13915-13932; incorporated herein by reference). Phenol 12 was hydroxyl-methylated using phenylboronic acid and para-formaldehyde to produce boronate ester 13 (Nagata, W.; Okada, K.; Aolki, T. Synthesis 1979, 365-368; incorporated herein by reference). Hydrolysis of 13 with hydrogen peroxide provided the requisite benzylic alcohol 14 which was converted to 3 in high yield after benzylation and deprotection. The ¹H NMR spectrum of synthetic 3 is consistent with the published data (Anam, E. M. Ind. J. Chem., Sect. B 1994, 33B, 1009-1011), but no ¹³C NMR is reported and we were unable to obtain an authentic sample for comparison The increased yield for the benzylation reaction relative to model compound 5 might be due to the decreased propensity of 4 to enolize and participate in undesirable side reactions.

While the antimicrobial activity of these compounds has been documented, little is known about their mechanism of action. Both 2 and 3 are potent inhibitors of E. coli GTPase activity (Table 3) and they exhibit IC₅₀ values similar to 1. These experiments indicate that the bacterial cell division protein FtsZ is a target of these compounds. Compound 11, which lacks the flavanone core structure, is much less potent.

TABLE 2
Inhibition of E. coli FtsZ GTPase activity by 1, 2, 3 and 11

| Compound | IC₅₀ (μM) |
|---|---|
| 1 (zantrin Z1)^{a} | 5.0 ± 0.5 |
| 2 (dichamanetin) | 12.5 ± 0.5 |
| 3 (2″′-hydroxy-5″-benzylisouvarinol-B) | 8.3 ± 0.5 |
| 11 | 60.4 ± 2.2 |

^{a}See Margalit et al. Proc. Nat'l. Acad. Sci. U.S.A. 2004, 101, 11821-11826; incorporated herein by reference.

In summary, we have developed the first efficient route to hydroxybenzylated flavanone natural products. We synthesized dichamanetin and 2′″-hydroxy-5″-benzyliso-uvarinol-B from a common core structure using a new zinc chloride-mediated benzylic coupling reaction. The efficient synthesis described herein will allow the preparation of a panel of derivatives so that the mechanism of action can be studied in more detail.

### Experimentals

General Methods: ¹H NMR spectra were obtained on a Varian Unity Inova 500 spectrometer (500 MHz). Chemical shifts (6) are reported in parts per million (ppm) relative to residual solvent (CHCl₃, s, δ 7.26 or CH₃COCH₃, δ 2.05). Multiplicities are given as: s (singlet), d (doublet), t (triplet), dd (doublet of doublets), m (multiplet), br s (broad singlet). Proton-decoupled ¹³C NMR spectra were obtained on a Varian Unity Inova 500 spectrometer (125 MHz). ¹³C chemical shifts are reported relative to CDCl₃ (t, δ 77.0) unless otherwise noted. IR frequencies are given in cm⁻¹ and spectra were obtained on a Perkin-Elmer Model 2000 FT-IR spectrophotometer. Tandem high performance liquid chromatography/mass spectral (LCMS) analyses were performed on a Micromass Platform LCZ mass spectrometer or a Micromass Platform LCT mass spectrometer in atmospheric pressure chemical ionization (APCI) mode after separation performed on a Waters Alliance 2690 separations module. The actual separations were performed on a Waters Symmetry® C₁₈ 3.5 μm, 2.1×50 mm column with a flow rate of 0.4 mL/min and a 12 min gradient of 15-100% CH₃CN in H₂O, with a constant 0.1% formic acid buffer using a Waters 996 photodiode array detector.

Silica gel chromatographic purifications were performed by flash chromatography with silica gel (EMD, 40-63 μm) packed in glass columns; the eluting solvent for each purification was determined by thin layer chromatography (TLC). Analytical TLC was performed on glass plates coated with 0.25 mm silica gel and visualized by ultraviolet light or by staining with cerric ammonium molybdate stain followed by gentle heating.

Manipulations under an inert atmosphere were carried out using standard Schlenk line techniques. Tetrahydrofuran (THF), dichloromethane (CH₂Cl₂), and toluene (PhCH₃) were dried by passage through a column of activated alumina (as described in Organometallics 1996, 15, 1518-1520). Microwave reactions were performed using Emrys™ Optimizer (Biotage, formerly Personal Chemistry) in a septa capped 0.5-2 mL Smith™ process vial with stirring. Unless otherwise specified, all commercially available reagents were used as received.

### Experimental Procedures:

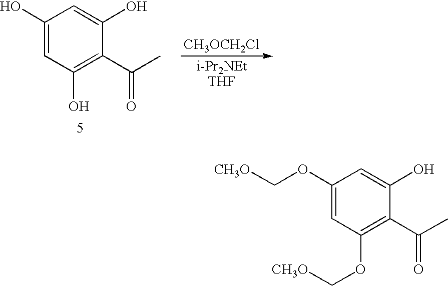

2′,4′-Bis(methoxymethoxy)-6′-hydroxyacetophenone. To a cooled (0° C.) solution of 2′,4′,6′-trihydroxyacetophenone monohydrate 5 (1.4 g, 7.5 mmol, 1.0 equiv) and N,N-diisopropylethylamine (13.1 mL, 75.0 mmol, 10.0 equiv) in 50 mL of dry THF was added dropwise chloromethylmethyl ether (1.3 mL, 16.5 mmol, 2.2 equiv) under an atmosphere of argon. The mixture was allowed to stir for 12 h after which it was quenched with 20 mL of water and extracted with EtOAc (3×25 mL). The combined organic layers were washed with 5% aqueous HCl, water, and brine, dried over anhydrous sodium sulfate, filtered and concentrated. The crude product was purified by flash column chromatography (0 to 15% EtOAc/hexanes) to yield the title compound as a white solid (1.4 g, 73%). The spectroscopic data (¹H and ¹³C NMR) were consistent with that reported in the literature. Choi et al., Heterocycles 1996, 43, 1223-1228; incorporated herein by reference.

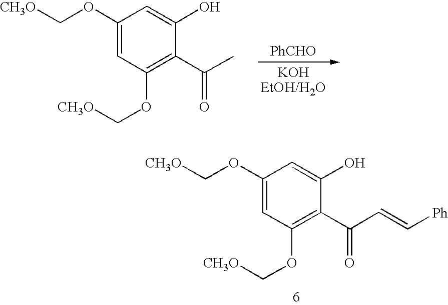

2′,4′-Bis(methoxymethoxy)-6′-hydroxychalcone (6). To a cooled (0° C.) solution of KOH (3.22 g, 57.6 mmol, 24.0 equiv) in EtOH—H₂O (1:1 v/v, 10 mL) was added dropwise a cooled (0° C.) mixture of benzaldehyde (0.25 mL, 2.45 mmol, 1.02 equiv) and 2′,4′-bis(methoxymethoxy)-6′-hydroxyacetophenone (0.614 g, 2.4 mmol, 1.0 equiv) in EtOH (5 mL). The reaction mixture was stirred at this temperature (0° C.) for 12 h and then for additional 36 h at rt. The mixture was then diluted with ether (20 mL) and the organic layer was separated. The aqueous layer was further extracted with ether (3×25 mL). The combined organic extracts were washed with 5% aqueous HCl, water, and brine solution, dried over Na₂SO₄, filtered, and concentrated. The crude product was purified by flash column chromatography (0 to 20% EtOAc/liexanes) to yield 6 (0.700 g, 85%) as an orange solid. The spectroscopic data (¹H and ¹³C NMR) were consistent with that reported in the literature. Litkei et al. Liebigs Ann. 1995, 1711-1715; incorporated herein by reference.

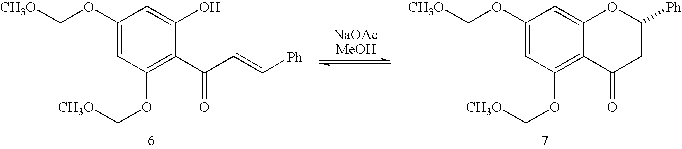

(±)-2′,4′-Bis(methoxymethoxy)flavanone (7). A solution of 6 (0.7 g, 2.0 mmol, 1.0 equiv) and anhydrous sodium acetate (2.25 g, 27.4 mmol, 13.7 equiv) in 400 mL of MeOH was refluxed under an atmosphere of argon for 48 h. After cooling, the solvent was removed in vacuo and H₂O (40 mL) was added to the resulting residue. The aqueous phase was extracted with EtOAc (4×50 mL). The combined organic layers were dried over anhydrous sodium sulfate, filtered, and concentrated. The crude product was purified by flash column chromatography (0 to 30% EtOAc/hexanes) to yield 7 (0.502 g, 72%) as a off-white solid (R_{F} 0.20 in 30% EtOAc in Hexanes) plus unreacted starting material 5 (0.198 g, 28%): ¹H NMR (500 MHz, CDCl₃) δ 7.44-7.37 (m, 5H), 6.44 (d, J=11.1 Hz, 2H), 5.43 (d, J=8.1 Hz, 1H), 5.27 (s, 2H), 5.17 (s, 2H), 3.53 (s, 3H), 3.47 (s, 3H), 3.04 (apparently dd, 1H), 2.81 (d, J=10.2 Hz, 1H); ¹³C NMR (125 MHz, CDCl₃) δ 189.0, 164.4, 163.2, 159.5, 138.7, 128.7, 128.6, 126.0, 107.3, 98.1, 97.4, 95.0, 94.0, 79.0, 56.5, 56.4, 45.8; IR (CDCl₃) 2958, 2905, 2828, 1680, 1573, 1436, 1148 cm⁻¹; MS (ESI-neg.) m/z calcd for C₁₉H₂₀O₆ (M+Na)⁺ 367.12, found 367.88.

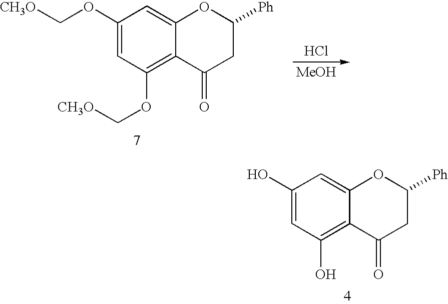

(±)-Pinocembrin (4). A solution of 7 (0.42 g, 1.2 mmol, 1.0 equiv) and 6N HCl (2.2 mL, 13.2 mmol, 11.0 equiv) in MeOH (10 mL) was refluxed for 30 min. After cooling, the solvent was removed in vacuo and the resulting residue was directly subjected to flash column chromatography (0 to 20% EtOAc/hexanes) to yield 4 (0.282 g, 90%) as a white solid. The spectroscopic data (¹H and ¹³C NMR) were consistent with that reported in the literature. Bick et al. Aust. J. Chem. 1971, 25, 449-451; incorporated herein by reference.

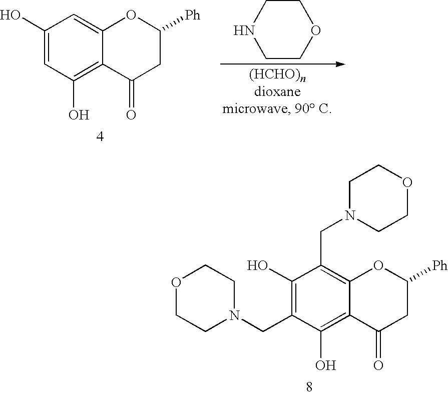

5,7-Dihydroxy-6,8-di((1-morpholino)-methyl)-flavanone (8). Pinocembrin 4 (70 mg, 0.27 mmol, 1.0 equiv), paraformaldehyde (33 mg, 1.09 mmol, 4.0 equiv), morpholine (58 μL, 0.65 mmol, 2.4 equiv), and anhydrous dioxane (2 mL) were mixed in a Smith™ process vial and sealed. The reaction mixture was heated in a microwave reactor at 90° C. for 10 minutes. After cooling to ambient temperature, the solvent was evaporated in vacuo. The crude product was subjected to flash column chromatography (EtOAc and then 20% MeOH/EtOAc) to yield 8 (90 mg, 73%) as a yellow oil: ¹H NMR (500 MHz, CDCl₃) δ 7.43-7.36 (m, 5H), 5.40 (dd, J=3.5, 9.5 Hz, 1H), 3.75 (m, 13H), 3.05 (dd, J=13.0, 4.5 Hz, 1H), 2.84 (dd, J=3.0, 14.0 Hz, 1H), 2.61-2.56 (two s, 8H); ¹³C NMR (125 MHz, CDCl₃) δ 195.5, 167.4, 161.5, 159.8, 138.6, 128.8, 128.6, 125.8, 101.6, 101.5, 101.0, 78.8, 66.6, 66.5, 52.8, 51.4, 51.3, 43.2; IR (CDCl₃) 3418, 2959, 1634, 1544 cm⁻¹; MS (ESI) m/z calcd for C₂₅H ₃₀N₂O₆ (M+H)⁺ 455.22, found 455.03.

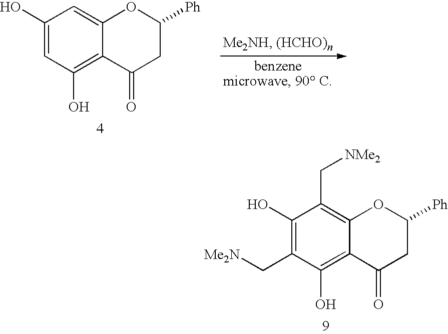

5,7-Dihydroxy-6,8-di((dimethylamino)-methyl)-flavanone (9). Pinocembrin 4 (14.5 mg, 0.057 mmol, 1.0 equiv), paraformaldehyde (4.1 mg, 0.136 mmol, 2.4 equiv), dimethylamine (2M in THF) (0.07 mL, 0.136 mmol, 2.4 equiv), and reagent grade benzene (2 mL) were mixed in a Smith™ process vial and sealed. The reaction mixture was heated in a microwave reactor at 90° C. for 10 minutes. After cooling to ambient temperature, the mixture was diluted with water (3 mL) and extracted with CH₂Cl₂ (3×10 mL). The organic layers were combined, washed with the brine solution (5 mL), dried over Na₂SO₄, and concentrated. The purification of this compound using silica gel column chromatography proved to be difficult due to high polarity. The LC-MS analysis and the crude ¹H NMR confirmed the presence of 9 and thus reactions with 9 were performed without further purification.

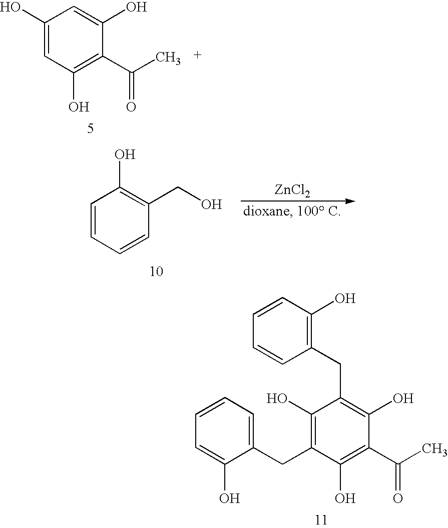

2,4,6-Trihydroxy-3,5-di(2′-hydroxybenzyl)-acetophenone (11). A solution of 2,4,6-trihydroxyacetophenone monohydrate 5 (200 mg, 1.07 mmol, 1.0 equiv), 2-hydroxybenzyl alcohol 10 (266 mg, 2.14 mmol, 2.0 equiv), and anhydrous ZnCl₂ (176 mg, 1.28 mmol, 1.2 equiv) in anhydrous dioxane (2 mL) was heated at 100° C. for 24 h under an atmosphere of argon. After cooling, the solvent was removed in vacuo and the crude product was purified by flash column chromatography (0 to 30% EtOAc/Hexanes) to afford 11 as a light yellow solid which on recrystallization with acetone/hexanes provided 11 as an off-white solid (200 mg, 49%), R_{F} 0.23 (30% EtOAc in Hexanes): ¹H NMR (500 MHz, CD₃COCD₃) δ 11.79 (br s, 1H), 9.72 (br s, 3H), 7.48 (d, J=7.5 Hz, 2H), 7.07 (t, J=7.5 Hz, 2H), 6.94 (d, J=8.0 Hz, 2H), 6.84 (t, J=7.5 Hz, 2H), 3.94 (s, 4H), 2.66 (s, 3H); ¹³C NMR (125 MHz, CD₃COCD₃) δ 205.5, 162.0, 161.6, 154.5, 133.1, 129.2, 129.1, 122.6, 116.6, 108.5, 107.2, 34.1, 24.0; IR (acetone) 3254, 1698, 1458, 1367, 1301, 1239, 1108, 755 cm⁻¹; MS (ESI-neg.) m/z calcd for C₂₂H₂₀O₆ (M−H)⁻ 379.12, found 379.03.

Procedure for microwave reactions: A solution of 2,4,6-trihydroxyacetophenone monohydrate 5 (200 mg, 1.07 mmol, 1.0 equiv), 2-hydroxybenzyl alcohol 10 (266 mg, 2.14 mmol, 2.0 equiv), and anhydrous ZnCl₂ (176 mg, 1.28 mmol, 1.2 equiv) in anhydrous dioxane (2 mL) were mixed in a Smith™ process vial and sealed. The reaction mixture was heated in a microwave reactor at 130° C. for 1 h. The solvent was removed in vacuo and the crude product was purified by flash column chromatography (0 to 30% EtOAc/Hexanes) to afford 11 as a light yellow solid which on recrystallization with acetone/hexanes provided 11 as an off-white solid (200 mg, 49%).

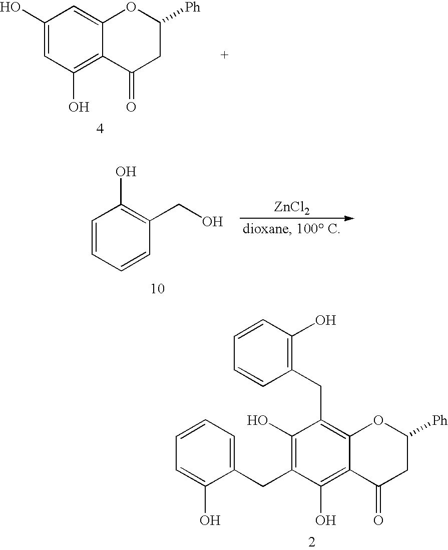

(±)-Dichamanetin (2). A solution of 4 (60 mg, 0.23 mmol, 1.0 equiv), 2-hydroxybenzyl alcohol 10 (61 mg, 0.48 mmol, 2.1 equiv), and anhydrous ZnCl₂ (67 mg, 0.48 mmol, 2.1 equiv) in anhydrous dioxane (2 mL) was heated at 100° C. for 24 h under an atmosphere of argon. After cooling, the solvent was removed in vacuo and the crude product was purified by flash column chromatography (0 to 30% EtOAc/Hexanes) to afford 2 (65 mg, 59%) as a light yellow solid, R_{F} 0.32 (30% EtOAc in Hexanes): ¹H NMR (500 MHz, CD₃COCD₃) δ 12.73 (s, 1H), 9.15 (br s, 2H), 7.59 (d, J=7.5 Hz, 2H), 7.46-7.38 (m, 3H), 7.23-7.22 (m, 1H), 7.09-7.07 (m, 1H), 7.04-6.99 (m, 2H), 6.86 (t, J=7.5 Hz, 2H), 6.77-6.69 (m, 2H), 5.64 (dd, J=2.5, 10.0 Hz, 2H), 3.92-3.91 (two s, 4H), 3.23 (dd, J=13.0, 4.0 Hz, 1H), 2.90 (dd, J=3.0, 14.0 Hz, 1H); ¹³C NMR (125 MHz, CD₃COCD₃) δ 198.7, 163.6, 161.7, 160.6, 155.6, 141.2, 132.3, 132.1, 130.6, 130.5, 130.4, 129.03, 128.99, 128.7, 128.67, 128.3, 122.1, 122.0, 116.9, 116.8, 109.6, 108.7, 104.5, 81.0, 44.6, 24.4, 23.7; IR (CDCl₃) 3427, 1631, 1489, 1457 cm⁻¹; MS (ESI) m/z calcd for C₂₉H₂₄O₆ (M+H)⁺ 469.17, found 469.98.

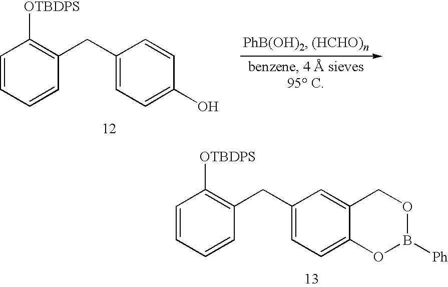

1-tert-Butyldiphenylsilyloxy-2′-hydroxymethyl-2,4′-methylene diphenol (13). A solution of 12 (416 mg, 0.95 mmol, 1.0 equiv), phenylboronic acid (307 mg, 1.05 mmol, 1.1 equiv), paraformaldehyde (286 mg, 9.5 mmol, 10.0 equiv), and propanoic acid (0.04 mL, 0.95 mmol, 1.0 equiv) in anhydrous benzene (10 mL) was refluxed in the presence of 4 Å molecular sieves for 24 h under an atmosphere of argon. Additional paraformaldehyde (60 mg) was added at intervals of 2 h. After cooling, the mixture was concentrated and 5 mL of H₂O was added. Extraction with ether (5×10 mL), washing with aqueous Na₂CO₃ and water, and evaporation of the solvent afforded a residue which was purified by flash column chromatography (0 to 10% EtOAc/Hexanes) to afford the dioxaborin 13 (411 mg, 78%) as a white solid, R_{F} 0.62 (30% EtOAc in Hexanes): ¹H NMR (500 MHz, CDCl₃) δ 7.98 (d, J=7.0 Hz, 2H), 7.69 (d, J=7.0 Hz, 4H), 7.51-7.35 (m, 9H), 7.12-7.02 (m, 3H), 6.85-6.81 (m, 3H), 6.49-6.47 (m, 1H), 5.17 (s, 2H), 4.14 (s, 2H), 1.02 (s, 9H); ¹³C NMR (125 MHz, CDCl₃) δ 153.5, 147.5, 136.1, 135.6, 134.5, 132.9, 131.6, 131.0, 130.9, 130.2, 129.3, 128.0, 127.9, 127.3, 125.1, 122.5, 121.2, 119.2, 118.0, 116.7, 63.2, 35.9, 26.7, 19.7; IR (CDCl₃) 3053, 3072, 2957, 2931, 2858, 1600, 1492, 1326, 1256 cm⁻¹; MS (ESI) m/z calcd for C₃₆H₃₅BO₃Si 554.24, found 554.98.

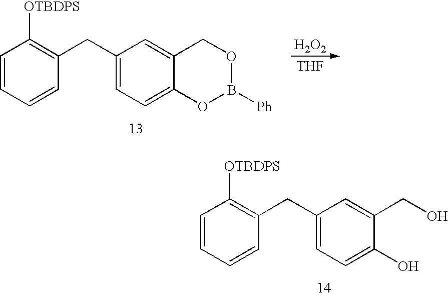

To a cooled (0° C.) solution of dioxaborin 13 (225 mg,) in dry THF (2 mL) was added 30% H₂O₂ solution (1 mL) dropwise. The reaction mixture was allowed to warm to rt in 4 h after which cold H₂O (2 mL) was added. The mixture was extracted with ether (10×10 mL). The combined extracts were washed with aqueous NaHSO₃, dried over anhydrous Na₂SO₄, and concentrated to give a residue which was purified by flash column chromatography (0 to 30% EtOAc/Hexanes) to afford 14 (188 mg, 99%) as a colorless viscous oil, R_{F} 0.41 (30% EtOAc in Hexanes): ¹H NMR (500 MHz, CDCl₃) δ 7.72 (d, J=8.0 Hz, 4H), 7.44-7.36 (m, 7H), 7.19 (br s, 1H), 7.12 (d, J=8.5 Hz, 1H), 7.06-7.04 (m, 1H), 6.92 (br s, 1H), 6.86-6.80 (m, 3H), 6.50-6.48 (m, 1H), 4.79 (s, 2H), 4.12 (s, 2H), 1.06 (s, 9H); ¹³C NMR (125 MHz, CDCl₃) δ 154.1, 153.2, 135.4, 132.7, 132.5, 131.1, 130.6, 129.9, 129.8, 128.2, 127.8, 126.9, 124.5, 120.9, 118.9, 116.4, 64.6, 35.3, 26.5, 19.4; IR (CDCl₃) 3403, 3071, 2931, 2857, 1490, 1254 cm⁻¹; MS (ESI) m/z calcd for C₃₀H₃₂O₃Si (M+Na)⁺ 491.20, found 491.00.

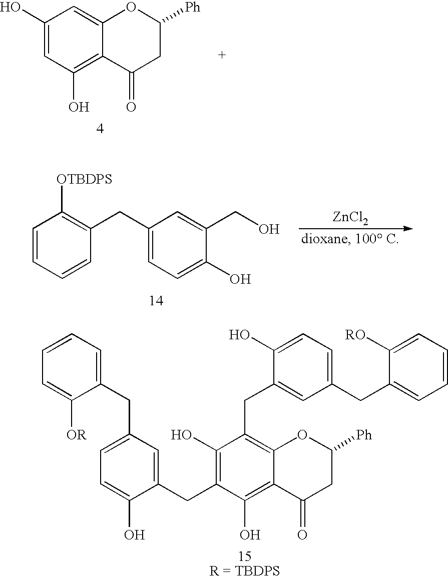

(±)-2′″,2″″-Bis(tert-butyldiphenylsilyloxy)-5″-benzylisouvarinol-B (15). A solution of 4 (90 mg, 0.35 mmol, 1.0 equiv), 14 (378 mg, 0.81 mmol, 2.3 equiv), and anhydrous ZnCl₂ (96 mg, 0.70 mmol, 2.0 equiv) in anhydrous dioxane (3 mL) was heated at 100° C. for 22 h under an atmosphere of argon. After cooling, the solvent was removed in vacuo and the crude product was purified by flash column chromatography (0 to 30% EtOAc/Hexanes) to afford 15 (374 mg, 92%) as a white solid, R_{F} 0.3 (30% EtOAc in Hexanes): ¹H NMR (500 MHz, CDCl₃) δ 12.63 (s, 1H), 9.97 (br s, 1H), 8.02 (br s, 1H), 7.78-7.74 (m, 8H), 7.47-7.33 (m, 19H), 7.09-7.08 (m, 1H), 6.96-6.73 (m, 9H), 6.53-6.62 (m, 2H), 5.42 (d, J=7.8 Hz, 1H), 4.17 (s, 2H), 4.04 (s, 2H), 3.98 (s, 2H), 3.91 (s, 2H), 3.14-3.07 (m, 1H), 2.84 (d, J=9.9 Hz, 1H), 1.07 (s, 9H), 1.06 (s, 9H); ¹³C NMR (125 MHz, CDCl₃) δ 196.4, 160.1, 159.5, 158.4, 153.2, 150.5, 150.3, 138.4, 135.4, 135.3, 133.7, 133.2, 132.7, 132.6, 132.5, 131.4, 131.3, 130.8, 130.3, 129.9, 129.8, 128.9, 128.3, 128.1, 127.7, 126.7, 126.6, 126.4, 126.2, 120.81, 120.78, 118.7, 118.6, 115.6, 115.5, 108.6, 107.3, 103.1, 79.5, 43.4, 35.4, 35.3, 26.45, 26.40, 23.3, 22.5, 19.4, 19.3; IR (CDCl₃) 3249, 3071, 2931, 2857, 1631, 1490, 1452, 1254, 1112, 909 cm⁻¹; MS (ESI-neg.) m/z calcd for C₇₅H₇₂O₈Si₂ (M−H)⁻ 1155.47, found 1155.56.

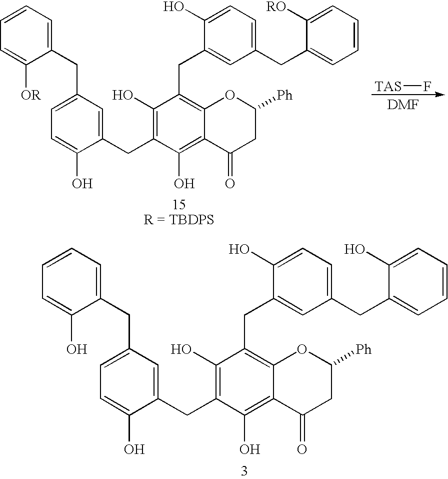

(±)-2′″-Hydroxy-5″-benzylisouvarinol-B⁵ (3). To a solution of 15 (65 mg, 0.056 mmol, 1.0 equiv) in anhydrous DMF (3 mL) at rt was added TAS-F (78 mg, 0.28 mmol, 5.0 equiv) under an atmosphere of argon. The reaction was stirred for 24 h at rt. The reaction mixture was diluted with EtOAc (15 mL) and washed with NaHCO₃ (5 mL), saturated LiCl (5×2 mL) and brine (5 mL) solution. The crude product was purified by flash column chromatography (0 to 50% EtOAc/Hexanes) to afford 3 as a light yellow solid which on recrystallization with CHCl₃/hexanes afforded a white solid (24.4 mg, 64%), R_{F} 0.42 (50% EtOAc in Hexanes): ¹H NMR (500 MHz, CDCl₃) δ 12.77 (s, 1H), 9.36 (br s, 1H), 7.44-7.42 (m, 6H), 7.19-7.07 (m, 4H), 6.95-6.89 (m, 3H), 6.83-6.80 (m, 3H), 6.73 (d, J=8.0 Hz, 2H), 6.64 (d, J=8.0 Hz, 1H), 5.33 (dd, J=2.5, 11.0 Hz, 2H), 5.06 (br s, 1H), 4.74 (br s, 1H), 3.91 (s, 2H), 3.81 (s, 2H), 3.74-3.72 (two s, 4H), 3.03 (dd, J=13.5, 3.5 Hz, 1H), 2.75 (dd, J=2.5, 14.5 Hz, 1H); ¹³C NMR (125 MHz, CDCl₃) δ 196.7, 161.5, 158.8, 158.6, 153.9, 153.8, 152.6, 150.9, 138.6, 132.6, 132.5, 132.2, 131.3, 130.9, 129.3, 129.2, 128.4, 128.3, 128.2, 128.0, 127.62, 127.59, 126.7, 126.6, 126.5, 121.6, 121.2, 116.9, 116.4, 115.9, 108.6, 107.7, 103.0, 79.9, 43.6, 36.6, 35.8, 23.8, 23.3; IR (CDCl₃) 3396, 2925, 1631, 1455, 908, 734 cm⁻¹; MS (ESI-neg.) m/z calcd for C₄₃H₃₆O₈ (M−H)⁻ 679.23, found 679.12.

¹H NMR Comparison

| Natural (2′″-Hydroxy-5″- | Synthetic (2′″-Hydroxy-5″- |
|---|---|
| benzylisouvarinol-B), ¹H NMR, | benzylisouvarinol-B), ¹H NMR, CDCl₃, |
| CDCl₃, 300 MHz | 500 MHz |
| Not reported | 12.77 (1H, s) |
| Not reported | 9.36 (1H, br s) |
| 6.63-7.8 (19H, m) | 7.44-7.42 (6H, m), 7.19-7.07 (4H, m), |
| | 6.95-6.89 (3H, m), 6.83-6.80 (3H, m), |
| | 6.73 (2H, d), 6.64 (1H, d) |
| 5.73 (1H, dd) | 5.33 (1H, dd) |
| Not reported | 5.06 (1H, br s) |
| Not reported | 4.74 (1H, br s) |
| 4.00, 3.97, 3.87, 3.80 (8H, 4s) | 3.91, 3.81, 3.74, 3.72 (8H, 4s) |
| 3.04 (1H, dd) | 3.03 (1H, dd) |
| 2.74 (1H, dd) | 2.75 (1H, dd) |

GTPase Assay: Untagged E. coli FtsZ was expressed from pET-3Z⁺ in BL21(λDE3) cells (RayChaudhuri, D.; Park, J. T. Nature 1992, 359, 251-254) and purified using a two-step ammonium sulfate fractionation (Romberg et al. J. Biol. Chem. 2001, 276, 11743-11753). FtsZ GTPase assay was performed essentially as described by Margalit et al. (Margalit et al. Proc. Natl. Acad. Sci. U.S.A. 2004, 101, 11821-11826). Briefly, 2 μM E. coli FtsZ in polymerization buffer (4-morpholinepropanesulfonic acid, pH 6.5, 50 mM KCl, 5 mM MgCl₂) was preincubated with or without varying concentrations of test compounds (in dimethyl sulfoxide, DMSO) such that the DMSO concentrations in the samples were ˜2%. The control tubes received 2% DMSO alone. After a 5-min preincubation at 25° C., the reactions were initiated by adding 0.5 mM GTP. Aliquots were withdrawn after 5, 15 and 25 ininute intervals and quenched with 50 mM EDTA before color development with the malachite green solution [2 volumes malachite green (0.8 mg/ml)/1 volume polyvinyl alcohol (23.2 mg/ml)/1 volume ammonium molybdate (57.2 mg/ml in 2H₂O:3HCl)/2 volumes H₂O]. IC₅₀ values were determined from the relative slopes of GTP hydrolysis at different inhibitor concentrations compared to the control with DMSO only.

| Compound | IC₅₀ (μM) |
|---|---|
| (±)-Dichamanetin (2) | 12.5 ± 0.5 |
| (±)-2″′-Hydroxy-5″-benzylisouvarinol-B (3) | 8.3 ± 0.5 |
| 2,4,6-Trihydroxy-3,5-di(2′-hydroxybenzyl)- | 60.4 ± 2.2 |
| acetophenone (11) | |

### Example 2

### Synthesis of Other Dichamanetin Analogs

### 2′-2′″-dimethyl-3″-3′″-dichlorodichamanetin

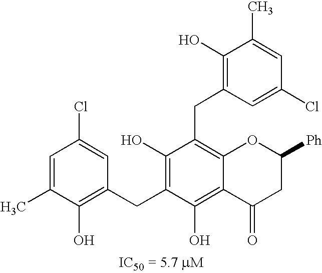

Pinocembrin (0.050 g, 0.19 mmol) and 3-chloro-2-hydroxy-5-methyl-benzyl alcohol (0.075 g, 0.43 mmol) were combined under the standard conditions to yield 0.083 g (75%) of 2″-2′″-dimethyl-3″-3′″-dichlorodichamanetin as a yellow solid. ¹H NMR (300 MHz, CD₃COCD₃) δ 12.8 (s, 1H), 7.62-7.64 (m, 2H), 7.38-7.51 (m, 3H), 7.17 (d, J=2.7 Hz, 1H), 7.04 (d, J=2.7 Hz, 1H), 6.83 (d, J=2.4 Hz, 2H), 5.40 (dd, J=9.6, 3.0 Hz, 1H), 3.64-3.80 (ni, 4H), 2.93 (dd, J=12.9, 3.9 Hz, 1H), (dd, J=13.8, 3.0 Hz, 1H), 2.13 (s, 3H), 2.12 (s, 3H).

### 6,8-bis(2-hydroxybenzyl)-flavone

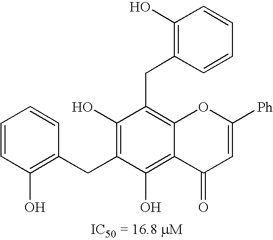

Chrysin (0.2 g, 0.78 mmol) and 2-hydroxybenzyl alcohol (0.196 g, 1.58 mmol) were combined under the standard conditions to yield 0.135 g (37%) of 6,8-bis(2-hydroxybenzyl)-flavone as a pale yellow solid. ¹H NMR (300 MHz, CD₃COCD₃) δ 13.5 (s, 1H), 9.25 (br s, 2H), 7.52-7.59 (m, 3H), 7.34-7.37 (m, 1H), 6.97-7.05 (m, 3H), 6.88-6.92 (m, 2H), 6.78-6.83 (m, 2H), 6.62-6.68 (m, 1H), 4.26 (s, 2H), 4.04 (s, 2H).

### 5,7-dihydroxy-6,8-bis(2-hydroxybenzyl)-2,2-dimethylchroman-4-one

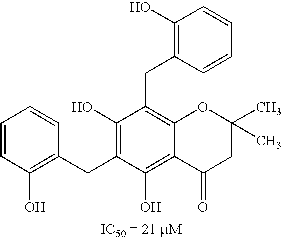

A solution of 5,7-dihydroxy-2,2-dimethylchroman-4-one (0.117 g, 561.9 mmol, 1.0 equiv; prepared by the method of Xie et al. (J. Med. Chem. 2001, 44, 664-671; incorporated herein by reference), 2-hydroxybenzyl alcohol (0.140 g, 1127.8 mmol, 2.0 equiv), and anhydrous ZnCl₂ (0.154 g, 1129.9 mmol, 2.0 equiv) in anhydrous dioxane (3 mL) was heated in a closed 5 mL glass vessel at 130° C. for 1 h under an atmosphere of argon. After cooling, the crude product was purified using flash cbromatography (0% to 30% EtOAc/hexanes), and the solvent was removed in vacuo to yield the product as a clear oil (0.195 g, 90%): ¹H NMR (500 MHz, CDCl₃) δ 12.5 (s, 1H), 8.50 (br s, 2H), 7.50 (dd, J=7.6, 1.6, 1H), 7.38 (dd, J=7.6, 1.6, 1H), 7.06 (apparent dd, J=7.8, 1.7, 2H), 6.87 (m, 2H), 6.80 (m, 2H), 3.89 (s, 1H), 3.83 (s, 1H), 2.68 (s, 2H), 1.50 (s, 6H); ¹³C NMR (125 MHz, CDCl₃) δ 196.8, 160.8, 159.4, 157.0, 152.3, 132.1, 131.7, 127.9, 127.8, 126.7, 126.5, 121.3, 121.1, 115.6, 115.6, 107.6, 107.3, 102.4, 79.3, 47.4, 26.8, 23.1, 22.4.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A compound of the formula: 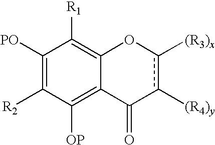
wherein
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; ═O; —C(═O)R_{A}; —CO₂R_{A}; —CN; —SCN; —SRA; —SORA; —SO₂R_{A}; —NO₂; —N(R_{A})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; ═O; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R^{C}; —CN; —SCN; —SRC; —SORC; —SO₂R_{C}; —NO₂; —N(R_{C})₂; —NHC(O)R_{C}; or —C(R_{C})₃; wherein each occurrence of R^{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; ═O; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of P is independently hydrogen, substituted or unsubstituted aliphatic, substituted or unsubstituted heteroaliphatic, a protecting group, substituted or unsubstituted acyl, substituted or unsubstituted aryl, or substituted or unsubstituted heteroaryl;
x is 1 or 2, wherein when the dashed line represents a bond, x is 1;
y is 1 or 2, wherein when the dashed line represents a bond, y is 1;
the dashed line represents the presence or absence of a bond; and
derivatives, salts, pro-drugs, and isomers thereof
with the proviso that when the dashed line represents no bond, R₃ is phenyl, and R₄ is hydrogen, then R₁ and R₂ are not both selected from the group consisting of:

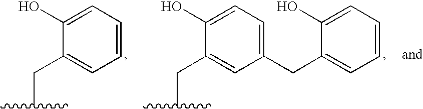

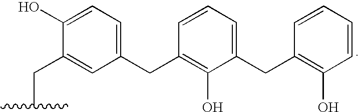

2. The compound of claim 1, wherein each occurrence of P is hydrogen.

3. The compound of claim 1, wherein each occurrence of P is hydrogen or an oxygen protecting group.

4. The compound of claim 1, wherein each occurrence of P is hydrogen or acyl.

5. The compound of claim 1, wherein each occurrence of P is hydrogen or alkyl.

6. The compound of claim 1, wherein R₁ is a substituted or unsubstituted arylaliphatic or heteroarylaliphatic moiety.

7. The compound of claim 1, wherein R₁ is a substituted or unsubstituted arylalkyl or heteroarylalkyl moiety.

8. The compound of claim 1, wherein R₁ is a substituted or unsubstituted benzyl moiety.

9. The compound of claim 1, wherein R₁ is a substituted benzyl moiety.

10. The compound of claim 1, wherein R₂ is a substituted or unsubstituted arylaliphatic or heteroarylaliphatic moiety.

11. The compound of claim 1, wherein R₂ is a substituted or unsubstituted arylalkyl or heteroarylalkyl moiety.

12. The compound of claim 1, wherein R₂ is a substituted or unsubstituted benzyl moiety.

13. The compound of claim 1, wherein R₂ is a substituted benzyl moiety.

14. The compound of claim 1, wherein R₁ and R₂ are the same.

15. The compound of claim 1, wherein R₁ and R₂ are different.

16. The compound of claim 1, wherein at least one occurrence of R₃ is a substituted or unsubstituted cyclic or heterocyclic moiety.

17. The compound of claim 1, wherein at least one occurrence of R₃ is a substituted or unsubstituted aryl or heteroaryl moiety.

18. The compound of claim 1, wherein at least one occurrence of R₃ is a substituted or unsubstituted phenyl moiety.

19. The compound of claim 1, wherein at least one occurrence of R₃ is an unsubstituted phenyl moiety.

20. The compound of claim 1, wherein at least one occurrence of R₄ is hydrogen.

21. The compound of claim 1, wherein all occurrences of R₄ are hydrogen.

22. The compound of claim 1, wherein at least one occurrence of R₄ is hydroxy.

23. The compound of claim 1, wherein at least one occurrence of 4 is alkoxy.

24. The compound of claim 1, wherein at least one occurrence of R₄ is C₁-C₆ alkyl.

25. The compound of claim 1, wherein at least one occurrence of R₄ is amino.

26. The compound of claim 1, wherein at least one occurrence of R₄ is substituted or unsubstituted aryl or heteroaryl.

27. The compound of claim 1, wherein the dashed line represents a bond.

28. The compound of claim 1, wherein the dashed line represents the absence of bond.

29. The compound of claim 1 of one of the formulae:

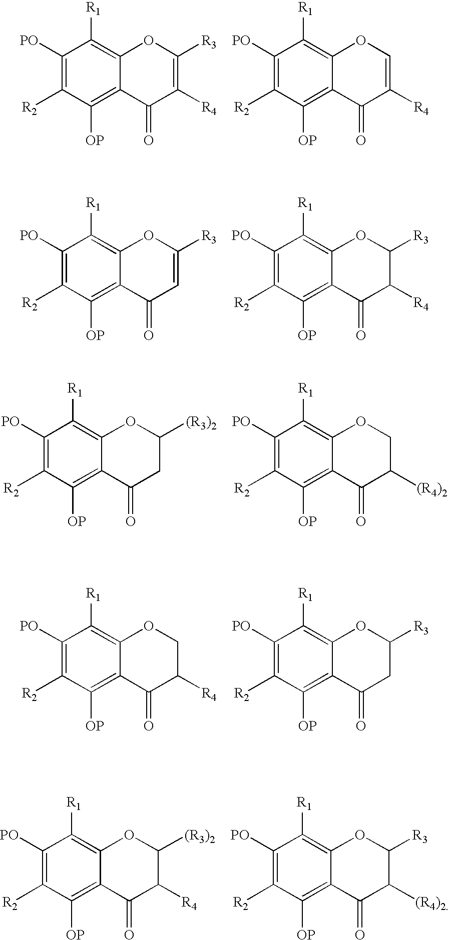

30. The compound of claim 1 of the formula: 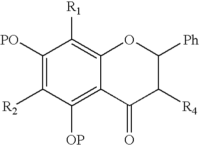
wherein
Ph is a substituted or unsubstituted phenyl moiety.

31. The compound of claim 1, wherein R₄ is hydroxy, alkoxy, or C₁-C₆ alkyl.

32. The compound of claim 1 of the formula: 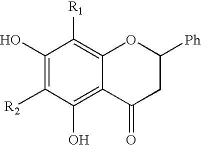
wherein
Ph is a substituted or unsubstituted phenyl moiety.

33. The compound of claim 32, wherein Ph is an unsubstituted phenyl moiety.

34. The compound of claim 32, wherein Ph is a substituted phenyl moiety.

35. The compound of claim 32, wherein Ph is a phenyl moiety substituted with a hydroxyl group.

36. The compound of claim 32 of the formula:

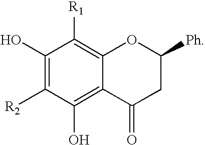

37. The compound of claim 32 of the formula:

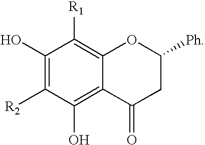

38. The compound of claim 1 of the formula: 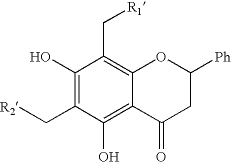
wherein
R₁′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or umbranched aryl; substituted or unsubstituted, branched or unbranched heteroaryl; —OR_{A}; —CN; —SCN; —SRA; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —CN; —SCN; —SR_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
Ph is a substituted or unsubstituted phenyl moiety.

39. The compound of claim 38, wherein R₁′ and R₂′ are independently substituted or unsubstituted aryl, or substituted or unsubstituted heteroaryl.

40. The compound of claim 38, wherein R₁′ and R₂′ are independently a monocyclic or bicyclic, substituted or unsubstituted heteroaryl or aryl ring system.

41. The compound of claim 38, wherein R₁′ and R₂′ are independently a monocyclic, substituted or unsubstituted heteroaryl or aryl ring system.

42. The compound of claim 38 of the formula:

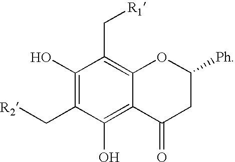

43. The compound of claim 38 of the formula:

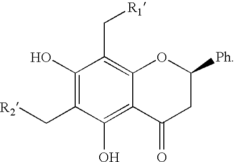

44. The compound of claim 38 of the formula: 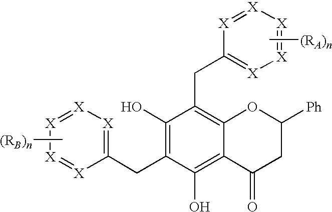
wherein
X is C or N and there are no more than three N atoms in any ring;
each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, diallylamino, heteroaryloxy; or heteroarylthio moiety; and
each occurrence of n is an integer between 1 and 5, inclusive.

45. The compound of claim 38 of the formula: 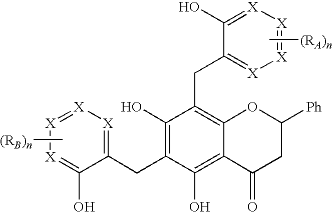
wherein
X is C or N and there are no more than three N atoms in any ring;
each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R_{B} is independently halogen; cyclic or acyclic, substituted or unsubstituted, branched or unbranched aliphatic; cyclic or acyclic, substituted or unsubstitulted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
each occurrence of n is an integer between 1 and 5, inclusive.

46. The compound of claim 38 of the formula: 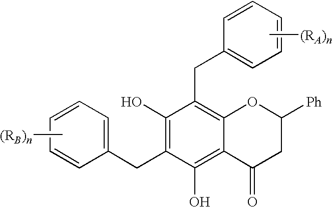
wherein
each occurrence of R_{A} is independently halogen; cyclic or acyclic, substituted or unsubstituted, branched or unbraiiched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; ═O; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
each occurrence of n is an integer between 1 and 5, inclusive.

47. The compound of claim 46 of the formula:

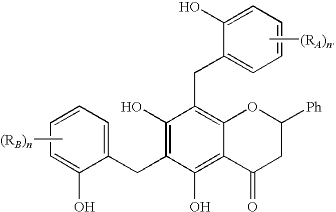

48. The compound of claim 46 of the formula:

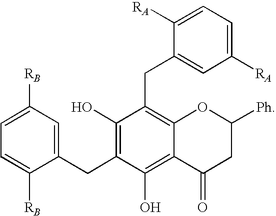

49. The compound of claim 1 of the formula:

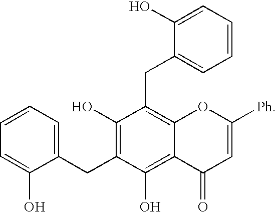

50. The compound of claim 1 of the formula:

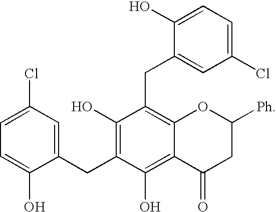

51. The compound of claim 1 of the formula:

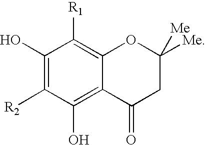

52. The compound of claim 1 of the formula:

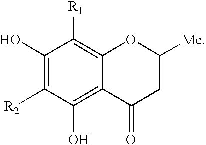

53. A method of preparing a compound of formula: 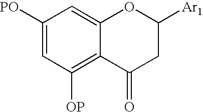
wherein
Ar₁ is a substituted or unsubstituted aryl or heteroaryl moiety; and
each occurrence of P is independently hydrogen, substituted or unsubstituted aliphatic, substituted or unsubstituted heteroaliphatic, a protecting group, substituted or unsubstituted acyl, substituted or unsubstituted aryl, or substituted or unsubstituted heteroaryl; the method comprising steps of:
protecting 2′,4′,6′-trihydroxyaceptophenone to yield a 2′,4′-bis(protected)-6′-hydroxyacetophenone of formula: 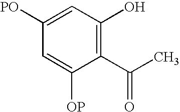
wherein P is substituted or unsubstituted aliphatic, substituted or unsubstituted heteroaliphatic, a protecting group, substituted or unsubstituted acyl, substituted or unsubstituted aryl, or substituted or unsubstituted heteroaryl;
condensing the 2′,4′-bis(protected)-6′-hydroxyacetophenone with an aldehyde of formula: 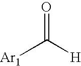
wherein Ar₁ is a substituted or unsubstituted aryl or heteroaryl moiety;
cyclizing the resulting 2′,4′-bis(protected)-6′-hydroxychalcone to yield a compound of formula:

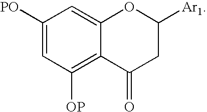

54. The method of claim 53, further comprising step of:
deprotecting to yield a compound of formula:

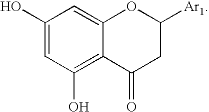

55. The method of claim 53, wherein P is methoxymethyl (MOM).

56. The method of claim 53, wherein Arl is a substituted or unsubstituted phenyl moiety.

57. The method of claim 53, wherein Arl is an unsubstituted phenyl moiety.

58. The method of claim 53, wherein the step of condensing is performed under basic conditions.

59. The method of claim 53, wherein the step of cyclizing is performed in the presence of NaOAc.

60. A method of preparing a compound of formula: 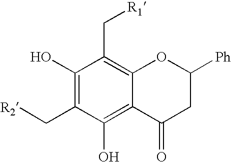
wherein
R₁′ is cyclic or acyclic, substituted or tmsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —CN; —SCN; —SRA; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂′ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —CN; —SCN; —SR_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
Ph is a substituted or unsubstituted phenyl moiety; the method comprising a step of:
benzylating a core stricture of formula: 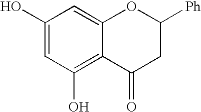
wherein Ph is a substituted or unsubstituted phenyl moiety, using a benzyl alcohol of formula: 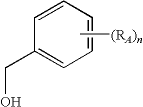
wherein
each occurrence of R_{A} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}′; ═O; —C(═O)R_{A}; —CO₂R_{A}′; —CN; —SCN; —SR_{A}′; —SOR_{A}′; —SO₂R_{A}′; —NO₂; —N(R_{A}′)₂; —NHC(O)R_{A}′; or —C(R_{A}′)₃; wherein each occurrence of R_{A}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
n is an integer between 1 and 5, inclusive.

61. The method of claim 60, wherein Ph is phenyl.

62. The method of claim 60, wherein the benzyl alcohol is of the formula:

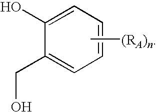

63. The method of claim 60, wherein the benzyl alcohol is of the formula: 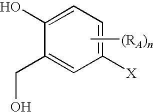
wherein X is a halogen.

64. The method of claim 60, wherein the benzyl alcohol is of the formula:

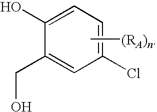

65. The method of claim 60, wherein the benzyl alcohol is of the formula:

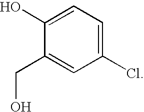

66. The method of claim 60, wherein the benzyl alcohol is of the formula: 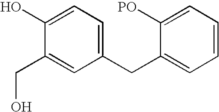
wherein P is hydrogen or an oxygen protecting group.

67. The method of claim 60, wherein P is TBDPS.

68. The method of claim 60, wherein the step of benzylating is performed in the presence of a Lewis or protic acid.

69. The method of claim 68, wherein the Lewis acid is ZnCl₂.

70. A pharmaceutical composition comprising a compound of claim 1 and a pharmaceutically acceptable excipient.

71. The pharmaceutical composition of claim 70 wherein the pharmaceutical composition is for the treatment of an infection.

72. The pharmaceutical composition of claim 71 wherein the infection is caused by gram positive bacteria.

73. A method of treating an infection in a subject, the method comprising steps of:
administering to the subject a therapeutically effective amount of a compound of claim 1.

74. The method of claim 73, wherein the infection is caused by bacteria.

75. The method of claim 73, wherein the infection is caused by gram positive bacteria.

76. A method of assaying a compound of claim 1 for antibiotic activity, the method comprising steps of:
contacting a compound of claim 1 with a bacterium; and
determining whether the compound kills or inhibits the growth of the bacterium.

77. The method of claim 76, wherein a plurality of compounds of claim 1 are screened.

78. The method of claim 76, wherein the bacteria are gram positive bacteria.

79. A method of screening a compound of claim 1 for anti-neoplastic activity, the method comprising steps of:
contacting a compound of claim 1 with a eukaryotic cell; and
determining whether the compound kills or inhibits the growth of the cell.

80. The method of claim 79, wherein the cell is derived from a cancer cell line.

81. The method of claim 79, wherein the cell is a human cell.

82. The method of claim 79, wherein the cell is a mammalian cell.

83. The method of claim 79, wherein the cell is a cancer cell.

84. The method of claim 79, wherein the cell is a normal cell.

85. The method of claim 79, wherein the cell is a non-cancerous cell.
